(19) United States
(12) Patent Application Publication
(10) Pub. No.: US 2013/0028962 A1
(43) Pub. Date: Jan. 31, 2013

(54) Triggered Cargo Release from Nanoparticle Stabilized Liposomes

(71) Applicants: Liangfang Zhang, San Diego, CA (US); Dissaya Pornpattananangkul, Bangkok (TH); Chun-Ming E. Huang, San Diego, CA (US)

(72) Inventors: Liangfang Zhang, San Diego, CA (US); Dissaya Pornpattananangkul, Bangkok (TH); Chun-Ming E. Huang, San Diego, CA (US)

(73) Assignee: THE REGENTS OF THE UNIVERSITY OF CALIFORNIA, Oakland, CA (US)

(21) Appl. No.: 13/607,094

(22) Filed: Sep. 7, 2012

Related U.S. Application Data
(63) Continuation of application No. PCT/US2011/028014, filed on Mar. 11, 2011.
(60) Provisional application No. 61/439,141, filed on Feb. 3, 2011.
(60) Provisional application No. 61/313,512, filed on Mar. 12, 2010.

(51) Int. Cl. A61K 9/127; A61K 31/327; A61K 38/14; A61P 31/00; A61P 35/00; A61P 17/00; A61K 31/20; A61P 31/04; B82Y 40/00; B82Y 5/00

(52) U.S. Cl. 424/450; 514/558; 514/714; 514/20.9; 977/773; 977/801; 977/907; 977/840

(57) ABSTRACT

Control of the fusion activity of liposomes by adsorbing biocompatible nanoparticles to the outer surface of phospholipid liposomes is disclosed. The biocompatible nanoparticles effectively prevent liposomes from fusing with one another. Release of cargo from the liposome is accomplished via trigger mechanisms that include pH triggers, pore forming toxing triggers and photosensitive triggers. Dermal drug delivery to treat a variety of skin diseases such as acne vulgaris and staph infections is contemplated.

### CROSS REFERENCES TO RELATED APPLICATIONS

This application is a continuation of PCT Application No. PCT/US2011/028014 filed on Mar. 11, 2011, which claims priority from U.S. Provisional Application Ser. No. 61/313,512 filed on Mar. 12, 2010, and U.S. Provisional Application Ser. No. 61/439,141 filed on Feb. 3, 2011, each of which is incorporated herein by reference in its entirety.

### STATEMENT REGARDING FEDERALLY SPONSORED RESEARCH OR DEVELOPMENT

This invention was made with Government support under National Institute of Health Grant Nos. U54CA119335, R01AI067395-01 and 1R21AI088147-01A1, and National Science Foundation Grant No. CMMI-1031239. The Government has certain rights in the invention.

### FIELD OF THE INVENTION

The present teachings relate to triggered cargo release from nanoparticle-liposome compositions and methods of use.

### BACKGROUND OF THE INVENTION

Using nanoparticles to differentially deliver therapeutic agents to the sites of action (also called targeted drug delivery) represents a central goal, a key challenge as well, of nanomedicine research. A common approach to reach this goal is to functionalize the surface of the nanoparticles with targeting ligands that specifically bind to the receptors over-expressed by the target cells. Various molecules have been demonstrated to bind to target cells including antibody, antibody fragments, aptamers, peptides, small molecules and so on. Although progress has been made to use ligands for active cellular targeting, none of the products have ever been approved for marketing a related therapeutic.

Liposomes are spherical lipid vesicles with a bilayer membrane structure consisting of amphiphilic lipid molecules and have been studied extensively for decades. There are a few liposome formulations that have been approved for marketing for therapeutic purposes, for example AmBisome (NeXstar Pharmaceuticals, San Dimas, USA), an FDA approved liposomal formulation of amphotericin B which has been widely used in the clinic to treat Candida spp., Aspergillus spp., Fusarium spp., and other fungi infections in neutropenic, visceral leishmaniasis, and methylmalonic acidaemia patients.

Despite these advantageous features of liposomes as a delivery vehicle, the applications of liposomes are usually limited by their instability due to uncontrollable fusion among liposomes, leading to short shelf-life, undesirable payload loss, and unexpected mixing. An extensively used approach to stabilize liposomes is to coat their surface with a “stealth” material such as polyethylene glycol (PEG). PEGylated liposomes can not only prevent liposomes from fusing with one another but also enhance their in vivo circulation lifetime by suppressing plasma proteins from adsorbing onto the liposome surface. Therefore, they have been widely used for systemic drug delivery. However, PEGylated liposomes are rarely used for topical drug delivery, especially to treat bacterial infections. This is mainly because the PEGylated coatings not only stabilize liposomes against fusion but also prevent them from fusing with bacterial membranes or prevent pore forming proteins such as toxins from accessing to the liposomes to release drug or other cargo payloads.

Therefore, it is desirable to develop new liposomes that are stabilized against fusion with one another before they are placed at the sites of action, including the manufacturing and storage periods, while their fusion activity can be activated once they are applied onto the target dermal sites. It is also desirable to develop liposomes that are stabilized against fusion with synthetic or biological membranes, but are accessible to pore forming proteins for controlled cargo release once they are applied onto the target skin sites.

### SUMMARY OF THE INVENTION

The present teachings include triggered drug release from stimuli-responsive nanoparticle-stabilized liposomes. In one embodiment, a liposome is provided that includes a lipid bilayer defining an inner sphere and an outer surface of the liposome, a plurality of biocompatible nanoparticles, the biocompatible nanoparticles connected to the lipid molecules with a stimuli-sensitive bond, and further comprising a cargo within the inner sphere, wherein said cargo is released upon triggering the stimuli-sensitive bond. In another embodiment, a liposome is provided that includes a lipid bilayer defining an inner sphere and an outer surface of the liposome, a plurality of biocompatible nanoparticles, said biocompatible nanoparticles being in contact with the lipid molecules via electrostatic interaction, and further comprising a cargo within the inner sphere, wherein said cargo is released upon triggering liposome pore formation.

In various aspects according to the embodiments above, the biocompatible nanoparticles can include gold nanoparticles, silver nanoparticles, and synthetic nanoparticles. In certain aspects, the surface of the biocompatible nanoparticles comprises anionic functional groups. In certain aspects, the surface of the biocompatible nanoparticles comprises cationic functional groups. In particular, the surface of the biocompatible nanoparticle can comprise carboxylates and chitosan.

In yet other aspects, the biocompatible nanoparticle is about 1 to about 20 nm in diameter. In various aspects, the liposome can comprise hydrogenated L-α-phosphatidylcholine and 1,2-di-(9Z-octadecenoyl)-3-trimethylammoniumpropane. In yet other aspects, the liposome comprises 50% cholesterol in the membrane and 100 mg/mL PEG in the solution.

In other aspects, the cargo is selected from the group consisting of antibiotics, antimicrobials, growth factors, chemotherapeutic agents, and combinations thereof. In particular, the cargo includes lauric acid, benzoyl peroxide, vancomycin, and combinations thereof.

In certain aspects, the liposome is about 10 to about 300 nm in diameter. In other aspects, the biocompatible nanoparticles comprise about 5 to about 25% of the liposome surface. In yet another aspect, the trigger can include dermal pH, naturally-occurring or synthetic toxin pore forming activity, and light administration. In various aspects, the stimuli-sensitive bond is a pH-sensitive bond. In yet other aspects, the trigger can be a pore forming toxin.

In another embodiment, a medicament delivery system is also provided comprising a liposome described above. In various aspects, the liposome can be delivered in a pharmaceutically acceptable vehicle.

In another embodiment, a method of selectively delivering cargo to target dermal sites is provided including administering a liposome described above to the target dermal site and triggering cargo release. In yet another embodiment, a method is provided for treating a dermal disease or condition including administering a therapeutically effective amount of a liposome described above to a target dermal site of a subject in need thereof and triggering cargo release. In yet another embodiment, a method is provided for treating a dermal disease or condition, the method comprising administering a therapeutically effective amount of a liposome described above to a subject in need thereof via the medicament delivery system described above. In yet another aspect, the condition of these methods can include MRSA infection, S. aureus infection, and P. acnes infection.

In yet another embodiment, a method is provided for stably storing medicaments prior to triggered release which includes enclosing the medicaments in a liposome described above.

These and other features, aspects and advantages of the present teachings will become better understood with reference to the following description, examples and appended claims.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. Schematic illustrations of carboxyl modified gold nanoparticles (AuC)-stabilized liposome and its destabilization at acidic pH. The liposome is stabilized by deprotonated AuC (Au—COO⁻) at neutral pH. When pH drops below the pKa value of carboxylic group (pKa˜5), Au—COO⁻ are protonated to form Au—COOH, which subsequently detach from the liposome, resulting in the formation of bare liposome with fusion activity resuming.

FIG. 2. Characterization of AuC-liposome by dynamical light scattering. (A) The size (diameter, m) and (B) surface zeta potential (mV) of bare liposomes and AuC-liposome with an AuC/liposome molar ratio of 200/1.

FIG. 3. Representative scanning transmission electron microscope (STEM) images showing the structure of AuC-liposome. (A) Secondary electron image shows that AuC nanoparticles adsorb on liposome surface. (B) Transmitted electron image of region shown in (A) further confirms the binding of AuC nanoparticles on liposome. (C) Dark field transmission image of AuC nanoparticles. (D) Transmission image of AuC nanoparticles.

FIG. 4. Fluorescence quenching and recovery yields of AuC-liposome at different AuC/liposome molar ratios (M_{AuC}/M_{L}) and different pH values. (A) AuC nanoparticles at different M_{AuC}/M_{L} molar ratio are allowed to adsorb to fluorescently labeled liposomes. Percentages of fluorescence quenching yield are plotted against M_{AuC}/M_{L} ratio. Inset: fluorescence emission spectra of AuC-liposome at different M_{AuC}/M_{L} ratio (from top to the bottom: 0, 22, 44, 66, 88, 110, 132, 154, 176, 200, 220, 240, 260, and 280). (B) Relative fluorescence recovery yield of AuC-liposome (M_{AuC}/M_{L}=200) at different pH values. Inset: fluorescence emission spectra of AuC-liposome at a series of pH values (from top to the bottom: 3, 3.5, 4, 4.5, 5, 7, 6.5, 6, and 5.5).

FIG. 5. UV-vis absorption spectra of AuC-liposome at pH=7 (top solid line) and pH=4 (bottom dashed line), respectively, after removal of unbound AuC through centrifugation. At pH=7, clear UV absorption spectrum of AuC was detected, indicating the strong binding of deprotonated AuC on liposome surface. At pH=4, negligible UV absorption of AuC was detected, indicating the detaching of protonated AuC from the liposome surface. Inset: AuC-liposome solutions after centrifugation to remove free AuC. Red color indicates the presence of AuC in the solution at pH=7.

FIG. 6. FRET measurement of AuC-mediated liposome fusion at pH=7 and pH=4, respectively. A florescent donor (C6NBD) and a fluorescent quencher (DMPE-RhB) were simultaneously incorporated into the anionic liposomes with a proper molar ratio that the quencher effectively quenched the fluorescence emission from the donor. The FRET-labeled anionic liposomes were then mixed with AuC-stabilized cationic liposomes. (A) Fluorescence emission spectra of C6NBD and DMPE-RhB with an excitation wavelength of 470 nm.

From top line to bottom line: AuC-cationic liposomes mixing with the anionic liposomes at pH=4 (dashed line); aqueous solution of the anionic liposomes alone without any gold nanoparticles or cationic liposomes at pH=7 (solid line); aqueous solution of the anionic liposomes alone without any gold nanoparticles or cationic liposomes at pH=4 (dashed line); AuB-cationic liopsomes mixing with the anionic liposomes at pH=4 (dashed line); AuB-cationic liopsomes mixing with the anionic liposomes at pH=7 (solid line); and AuC-cationic liposomes mixing with the anionic liposomes at pH=7 (solid line); (B) A zoom in of fluorescence emission spectra of C6NBD (donor) at different conditions from panel (A) where the top two lines are AuB, the middle two lines are AuC and the bottom two lines are aqueous solutions; (C) Relative fusion activity of AuC-cationic liposomes with anionic liposomes in contrast to AuB-cationic liposomes at pH=7 and pH=4, respectively.

FIG. 7. Schematic illustrations of carboxyl modified gold nanoparticles (AuC)-stabilized LipoLA and its destabilization at acidic pH.

FIG. 8. UV-vis absorption spectra of AuC—Mg-lipoLA at pH=7 (solid line) and pH=4 (dashed line), respectively, after removal of unbound AuC through centrifugation. At pH=7, clear UV absorption spectrum of AuC was detected, indicating the strong binding of deprotonated AuC on liposome surface. At pH=4, negligible UV absorption of AuC was detected, indicating the detaching of protonated AuC from the liposome surface. Inset: AuC—Mg-lipoLA solutions after centrifugation to remove free AuC. Purple color indicates the presence of AuC in the solution at pH=7.

FIG. 9. Interaction between AuC—Mg-lipoLA and P. acnes from pH=4 to pH=7. Emission spectra of P. acnes (7.93×10⁸ CFU/mL) after incubation with RhB labeled AuC—Mg-LipoLA (140 μg/mL of initial lipid concentration) at pH range from 4 to 7 with removal of excess RhB—AuC—Mg-LipoLA.

FIG. 10. Antimicrobial activity of AuC—Mg-LipoLA against P. acnes. (A) AuC—Mg-LipoLA were incubated with P. acnes (5×10⁷ CFU/mL) at different pHs for 10 min under anaerobic conditions. The results showed that at pH 4.0, AuC—Mg-LipoLA completely killed P. acnes. Incubation of P. acnes with empty liposome solution (without LA) and buffer at pH 4 served as negative controls. (B) At pH=4, AuC—Mg-LipoLA were incubated with P. acnes for 0, 5, 7.5, and 10 min respectively. Data represent mean±SD of three individual experiments. UD: undetectable.

FIG. 11. Schematic principle of bacterial toxin-triggered antibiotic release from gold nanoparticle-stabilized liposomes to treat toxin-secreting bacteria. Vancomycin-loaded liposomes are protected by absorbing chitosan-coated gold nanoparticles (AuChi) onto their surface to prevent them from fusing with one another or with bacterial membranes. Once the AuChi-stabilized liposomes (AuChi-Liposome) encounter bacterial toxins, the toxins form pores in the liposome membranes and thus release the encapsulated antibiotics, which subsequently kill or inhibit the growth of the bacteria that secrete the toxins.

FIG. 12. Synthesis and characterization of AuChi and AuChi-Liposome. (A) ¹H-NMR spectra of chitosan and AuChi, indicating the coating of chitosan on the surface of gold nanoparticles. (B) UV-Vis absorption spectrum of AuChi. Insets: representative secondary electron image (SEI) of AuChi and transmitted electron image (TEI) of the inner gold nanoparticles of AuChi. (C) The surface zeta potential (mV) of bare liposome (without AuChi) and AuChi-Liposome with a liposome/AuChi molar ratio of 1:300.

FIG. 13. Fusion ability of AuChi-Liposome at different liposome/AuChi molar ratios. The fluorescent dyes, ANTS and DPX, were encapsulated inside the liposomes at a concentration that DPX maximally quenched the fluorescence of ANTS. Upon fusion with bare liposomes (without AuChi or dyes), the fluorescence of ANTS recovered due to the dilution of the dyes. (A) The measured fluorescence emission spectra of ANTS after incubating ANTS/DPX loaded liposomes in PBS (serving as background fluorescence signal) and in 0.1% Triton X-100 (serving as maximal fluorescence signal), respectively, for 1 h at room temperature. (B) AuChi-Liposome with a liposome/AuChi molar ratio of 1:0, 1:150, or 1:300 were mixed with bare liposomes (without AuChi or dyes) at a molar ratio of 1:4. After incubation for 1 h at room temperature, the bare liposomes were broken by fusing with a centrifugal filter unit. The resulting fluorescence emission intensity of ANTS in the filtrate at 510 nm was measured.

FIG. 14. Toxin-induced pore forming in liposome membranes at various concentrations of cholesterol and PEG. (A) Liposomes with 0, 10, 25, and 50% (w/w) cholesterol were incubated with 20 μg/mL α-toxin for 1 h at room temperature. The dyes released from the pores were quantified by measuring fluorescence emission intensity of ANTS at 510 nm. Percentage of pore forming was obtained by comparing the α-toxin induced dye release with complete dye release caused by 1% (v/v) Triton-X-100. (B) Liposomes with 50% (w/w) cholesterol were incubated with 20 μg/mL α-toxin for 1 h at room temperature in the presence of various concentrations of PEG molecules (Mn=2000 Da), ranging from 0 to 150 mg/mL.

FIG. 15. Accumulative vancomycin release profile from vacomycin-loaded AuChi-Liposome after incubation with MRSA bacteria (1×10⁸ CFU/mL) for 0.5 h and 24 h, respectively. The released vancomycin was quantified by reversed phase HPLC. The corresponding samples incubated with PBS (without MRSA bacteria) were used as negative controls. Inset: the linear calibration standard curve of vancomycin at various concentrations measured by HPLC.

FIG. 16. Antimicrobial activity of vancomycin AuChi-Liposome against MRSA bacteria. Vancomycin AuChi-Liposome were incubated with MRSA bacteria (1×10⁸ CFU/mL) in 5% TSB for 24 h in the presence of 100 mg/mL PEG. The toxins secreted by the bacteria form pores in the AuChi-Liposome and release the encapsulated vancomycin, which subsequently inhibits the growth of the bacteria. The bacterial growth rate was determined by measuring absorbance at 600 nm after incubation. Vancomycin liposome (without AuChi) and free vancomycin with the same drug concentration (62 μg/mL) served as positive controls. AuChi-Liposome (without vancomycin) and PBS served as negative controls. Data represent mean±SD (n=3).

### DETAILED DESCRIPTION OF THE INVENTION

### Abbreviations and Definitions

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

In the descriptions of molecules and substituents, molecular descriptors can be combined to produce words or phrases that describe substituents. Such descriptors are used in this document. Examples include such terms as aralkyl (or arylalkyl), heteroaralkyl, heterocycloalkyl, cycloalkylalkyl, aralkoxyalkoxycarbonyl and the like. A specific example of a compound encompassed with the latter descriptor aralkoxyalkoxycarbonyl is C₆H₅—CH₂—CH₂—O—CH₂—O—C(O) wherein C₆H₅ is phenyl. It is also to be noted that a substituents can have more than one descriptive word or phrase in the art, for example, heteroaryloxyalkylcarbonyl can also be termed heteroaryloxyalkanoyl. Such combinations are used herein in the description of the compounds and methods of this invention and further examples are described herein.

Anionic: The term “anionic” as used herein refers to substances capable of forming ions in aqueous media with a net negative charge.

Anionic functional group: The term “anionic functional group” as used herein refers to functional group as defined herein which possesses a net negative charge. Representative anionic functional groups include carboxylic, sulfonic, phosphonic, their alkylated derivatives, and so on.

Cationic: The term “cationic” as used herein refers to substances capable of forming ions in aqueous media with a net positive charge.

Carboxylate: The term “carboxylate” as used herein refers to the —CO₂—.

Functional group: The term “functional group” as used herein, refers to a chemical group that imparts a particular function to an article (e.g., nanoparticle) bearing the chemical group. For example, functional groups can include substances such as antibodies, oligonucleotides, biotin, or streptavidin that are known to bind particular molecules; or small chemical groups such as amines, carboxylates, and the like.

Liposome: The term “liposome” as used herein refers to unilamellar or multilamellar lipid vesicles which enclose a fluid space. The walls of the vesicles, also referred to as a membrane, are formed by a bimolecular layer of one or more lipid components (e.g., multiple phospholipids plus cholesterol) having polar heads and non-polar tails, such as a phospholipid. In an aqueous (or polar) solution, and in a unilamellar liposome, the polar heads of one layer orient outwardly to extend into the surrounding medium, and the non-polar tail portions of the lipids associate with each other, thus providing a polar surface and a non-polar core in the wall of the vesicle. In a multilamellar liposome, the polar surface of the vesicle also extends to the core of the liposome and the wall is a bilayer. The wall of the vesicle in either of the unilamellar or multilamellar liposomes can be saturated or unsaturated with other lipid components, such as cholesterol, free fatty acids, and phospholipids. In such cases, an excess amount of the other lipid component can be added to the vesicle wall which will shed until the concentration in the vesicle wall reaches equilibrium, which can be dependent upon the liposome environment. Liposomes may also comprise other agents that may or may not increase an activity of the liposome. For example, polyethylene glycol (PEG) can be added to the membrane to enhance pore formation. In addition, biocompatible nanoparticles are added to the membrane to stabilize the liposome as described herein.

Medicament: The term “medicament” as used herein refers to a substance, formulation or device that treats or prevents or alleviates the symptoms of disease or condition in a patient or subject to whom the medicament is administered.

Nanoparticle: The term “nanoparticle” as used herein refers to a particle having a diameter of between about 1 nm and about 1000 nm. Similarly, by the term “nanoparticles” is meant a plurality of particles having an average diameter of between about 1 nm and about 1000 nm. The term includes biocompatible nanoparticles that can be biodegradable, cationic nanoparticles including, but not limited to, gold, silver, and synthetic nanoparticles that stabilize liposomes according the examples provided below. An example of a biocompatible synthetic nanoparticle includes polystyrene and the like.

Pharmaceutically acceptable: The terms “pharmaceutically acceptable” as used herein means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

Pharmaceutically acceptable salt: The terms “pharmaceutically acceptable salt” as used herein refer to acid addition salts or base addition salts of the compounds in the present disclosure. A pharmaceutically acceptable salt is any salt which retains the activity of the parent compound and does not impart any deleterious or undesirable effect on a subject to whom it is administered and in the context in which it is administered. Pharmaceutically acceptable salts include, but are not limited to, metal complexes and salts of both inorganic and carboxylic acids. Pharmaceutically acceptable salts also include metal salts such as aluminum, calcium, iron, magnesium, manganese and complex salts. In addition, pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycolic, glycolylarsanilic, hexamic, hexylresorcjnoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zurich, 2002; Berge et al., J. Pharm. Sci. 66: 1, 1977).

Pharmaceutically acceptable carrier: The terms “pharmaceutically acceptable carrier” as used herein refers to an excipient, diluent, preservative, solubilizer, emulsifier, adjuvant, and/or vehicle with which a compound is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when a compound is administered intravenously. Saline solutions and aqueous dextrose and glycerol solutions may also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A compound, if desired, may also combine minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compounds in combination with carriers are known to those of skill in the art.

Phospholipid: The term “phospholipid”, as used herein, refers to any of numerous lipids contain a diglyceride, a phosphate group, and a simple organic molecule such as choline. Examples of phospholipids include, but are not limited to, Phosphatidic acid (phosphatidate) (PA), Phosphatidylethanolamine (cephalin) (PE), Phosphatidylcholine (lecithin) (PC), Phosphatidylserine (PS), and Phosphoinositides which include, but are not limited to, Phosphatidylinositol (PI), Phosphatidylinositol phosphate (PIP), Phosphatidylinositol bisphosphate (PIP2) and Phosphatidylinositol triphosphate (PIP3). Additional examples of PC include DDPC, DLPC, DMPC, DPPC, DSPC, DOPC, POPC, DRPC, and DEPC as defined in the art.

Cargo: The term “cargo”, as used herein, refers to agents that are therapeutically active when in a dermal environment, e.g., on the epidermis, epidermal wound, acne lesions, and the scalp. Such cargo includes, but is not limited to, antibiotics, antimicrobials, growth factors, benzoyl peroxide, chemotherapeutics, and other agents that affect the target dermal condition such as medicaments described above. For example, vancomycin can be used to treat MRSA as described below when administered to the epidermis or epidermal wound.

Pore Forming Toxins: The term “pore forming toxins”, as used herein, refers to molecules that open unregulated channels in the membranes of target cells. Examples of naturally occurring pore forming molecules include, but are not limited to, alpha toxin, beta toxin, delta toxin, gamma toxin, and aflatoxin. Examples of synthetic pore forming toxins include surfactants, such as Triton-X 100®. Those of skill in the art will recognize other naturally occurring and synthetic pore forming toxins.

Triggered Cargo Release from Nanoparticle Stabilized Liposomes

The present invention provides stimuli-responsive biocompatible nanoparticle-stabilized liposomes and triggered cargo release therefrom. Such liposomes can selectively deliver a cargo including, but not limited to, antibiotics, antimicrobials, and other therapeutic agents, to dermal targets. Cargo delivery is performed selectively by activation of one or more triggers including, but not limited to, dermal pH, naturally-occurring or synthetic toxin pore forming activity, and photosentitive triggers (e.g., via administration of an external UV light source). When such triggers are not present, the liposome cargo is not actively released. Therefore, such liposomes also have the advantage of preventing unwanted cargo release from the liposomes prior to triggering.

In one embodiment, the invention provides a liposome comprising a lipid bilayer defining an inner sphere and an outer surface of the liposome, a plurality of biocompatible nanoparticles, the biocompatible nanoparticles connected to the lipid molecules with a stimuli-sensitive bond, and further comprising a medicament within the inner sphere. In certain aspects, the plurality of biocompatible nanoparticles may be bound to hydrophyllic heads of lipid molecules of the lipid bilayer. In certain aspects, the lipid molecules may include phospholipids. In particular, lipid molecules that may can be part of the lipid membrane include hydrogenated L-α-phosphatidylcholine and 1,2-di-(9Z-octadecenoyl)-3-trimethylammonium-propane. In particular, the lipid molecules may comprise hydrogenated L-α-phosphatidylcholine, lauric acid, and magnesium sulfate. In certain aspects, the biocompatible nanoparticles may be connected to the lipid molecules with a pH sensitive bond. In certain aspects, the outer surface of the biocompatible nanoparticle may comprise anionic functional groups. In particular, the anionic functional group may be carboxylate. In certain aspects, the biocompatible nanoparticle is from about 1, 2, 3, 4, 5, 6, 7, 8, 9, 10, 11, 12, 13, 14, 15, 16, 17, 18, 19 or about 20 nm in diameter. In particular, the biocompatible nanoparticle is gold and about 4 nm in diameter. In certain aspects, the outer surface of the liposome comprises cationic functional groups. In yet other aspects, the liposome can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 400, or 500 nm in diameter. In particular, the liposome is about 88 nm in diameter. In certain aspects, the biocompatible nanoparticles are integral to (e.g., measured by the surface area) from about 5, 6, 7, 8, 9, 10, 11, 12, 13, 14, 15, 16, 17, 18, 19, 20, 21, 22, 23, 24 or 25% of the liposome surface. In particular, the biocompatible nanoparticles are bound to about 14% of the liposome outer surface.

For example, a liposome is provided in which biocompatible nanoparticles are bound to the surface of the liposome that stabilizes, or prevents the fusion of one liposome to another liposome, at neutral pH. Such biocompatible nanoparticles (e.g., having a diameter of ˜4 nm) can be biodegradable, cationic nanoparticles including, but not limited to, gold, silver, and synthetic nanoparticles that stabilize liposomes (e.g., having a diameter of ˜100 nm) according to the Examples provided below. The bound biocompatible nanoparticles can detach from the liposomes when the environment acidity increases to about pH<5, resulting in the formation of bare liposomes that can actively fuse with various biological membranes. It has been well documented that human skin is typically acidic (pH=3.9˜6.0), especially the infectious lesions on the skin. For example, the pH value is about 4.0 at the acne lesions and 4.5-6.3 at comedones. Therefore acid-responsive liposomes with tunable fusion ability are provided for dermal cargo delivery.

In another embodiment, the invention provides a liposome comprising a lipid bilayer defining an inner sphere and an outer surface of the liposome, a plurality of biocompatible nanoparticles, biocompatible nanoparticles being in contact with the lipid molecules via electrostatic interaction, and further comprising a medicament within the inner sphere. In certain aspects, the lipid molecules may include phospholipids. In particular, lipid molecules that may can be part of the lipid membrane include hydrogenated L-α-phosphatidylcholine and 1,2-di-(9Z-octadecenoyl)-3-trimethylammonium-propane. In particular, the lipid molecules may comprise hydrogenated L-α-phosphatidylcholine, cholesterol, and polyethylene glycol. In certain aspects, the outer surface of the biocompatible nanoparticle may comprise cationic functional groups. In particular, the cationic functional group may be chitosan. In certain aspects, the biocompatible nanoparticle is from about 1, 2, 3, 4, 5, 6, 7, 8, 9, 10, 11, 12, 13, 14, 15, 16, 17, 18, 19 or about 20 nm in diameter. In particular, the biocompatible nanoparticle is gold and about 4 nm in diameter. In certain aspects, the outer surface of the liposome comprises anionic functional groups. In yet other aspects, the liposome can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 400, or 500 nm in diameter. In particular, the liposome is about 88 nm in diameter. In certain aspects, the biocompatible nanoparticles are integral to (e.g., measured by the surface area) from about 5, 6, 7, 8, 9, 10, 11, 12, 13, 14, 15, 16, 17, 18, 19, 20, 21, 22, 23, 24 or 25% of the liposome surface. In particular, the biocompatible nanoparticles are bound to about 14% of the liposome outer surface.

In another example, a liposome is provided that can selectively deliver cargo to targeted dermal sites which is triggered by pore forming toxins. In various aspects, the pore forming toxin opens pores in the liposome to release cargo at a targeted dermal site. In such case, the biocompatible nanoparticle does not necessarily detach from the liposome membrane to deliver its cargo. Once the liposomes contact bacteria that secrete toxins, they provide for the toxins to insert into the liposome membranes and form pores, through which the nanoparticle-stabilized liposomes release therapeutic agents. The released drugs subsequently impose antimicrobial effects on the toxin-secreting bacteria. Using Methicillin-resistant Staphylococcus aureus (MRSA) as a model bacterium and vancomycin as a model anti-MRSA antibiotic, the Examples herein demonstrate that the synthesized nanoparticle-stabilized liposomes can completely release the encapsulated vancomycin within 24 h in the presence of MRSA bacteria and lead to inhibition of MRSA growth as effective as an equal amount of vancomycin loaded liposomes (without nanoparticle stabilizers) and free vancomycin.

In various aspects, the present invention includes a medicament delivery system comprising the liposomes described above. In certain aspects, the medicament may be formulated with a pharmaceutically acceptable vehicle.

In accordance with yet another aspect of the present invention, a method for treating a disease is provided, the method comprising administering a therapeutically effective amount of a cargo via the medicament delivery system described above, to a patient in need thereof. In certain aspects, the drug may be benzoyl peroxide or lauric acid. In certain aspects, the disease may be skin disease. In particular, the skin disease may be P. acnes infection or S. aureus infection.

In various aspects, the present invention includes a process for preparing the liposomes described above. The process involves combining biocompatible nanoparticles with liposomes. In certain aspects, the surface of the biocompatible nanoparticle may comprise anionic functional groups or cationic functional groups. In particular, the surface of the biocompatible nanoparticle may comprise carboxylates including chitosan. In certain aspects, the biocompatible nanoparticle may be gold, and about 1 to about 20 nm in diameter. In particular, the biocompatible nanoparticle may be about 4 nm in diameter. In certain aspects, the liposome may comprise phospholipids. In particular, the liposome may comprise hydrogenated L-α-phosphatidylcholine and 1,2-di-(9Z-octadecenoyl)-3-trimethylammonium-propane. In particular, the liposome may comprise hydrogenated L-α-phosphatidylcholine, lauric acid, and magnesium sulfate. In particular, the lipid molecules may comprise hydrogenated L-α-phosphatidylcholine, cholesterol, and polyethylene glycol. In certain aspects, the biocompatible nanoparticle is from about 1, 2, 3, 4, 5, 6, 7, 8, 9, 10, 11, 12, 13, 14, 15, 16, 17, 18, 19 or about 20 nm in diameter. In particular, the biocompatible nanoparticle is gold and about 4 nm in diameter. In certain aspects, the outer surface of the liposome comprises anionic functional groups. In yet other aspects, the liposome can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 400, or 500 nm in diameter. In particular, the liposome is about 88 nm in diameter. In certain aspects, the biocompatible nanoparticles are integral to (e.g., measured by the surface area) from about 5, 6, 7, 8, 9, 10, 11, 12, 13, 14, 15, 16, 17, 18, 19, 20, 21, 22, 23, 24 or 25% of the liposome surface. In particular, the biocompatible nanoparticles are bound to about 14% of the liposome outer surface.

Methods of stably storing medicaments and cargo are provided. As described above, prior to triggered cargo release, cargo can be stably stored.

### Triggered Cargo Release Via Nanoparticle Detachment

The present invention provides stimuli-responsive biocompatible nanoparticles-stabilized liposomes in which biocompatible nanoparticles bind to the surface of liposomes and thus stabilize the liposomes at neutral pH. The bound biocompatible nanoparticles detach from the liposomes when the environment acidity increases to about pH<5, resulting in the formation of bare liposomes that can actively fuse with various biological membranes. Human skin is typically acidic (pH=3.9˜6.0), especially the infectious lesions on the skin. For example, the pH value is about 4.0 at the acne lesions and 4.5-6.3 at comedones. Therefore acid-responsive liposomes with tunable fusion ability is effective for dermal cargo delivery. See, e.g., Pornpattananangkul, D. et al. ACS Nano 2010, 4, 1935-1942, incorporated herein by reference in its entirety.

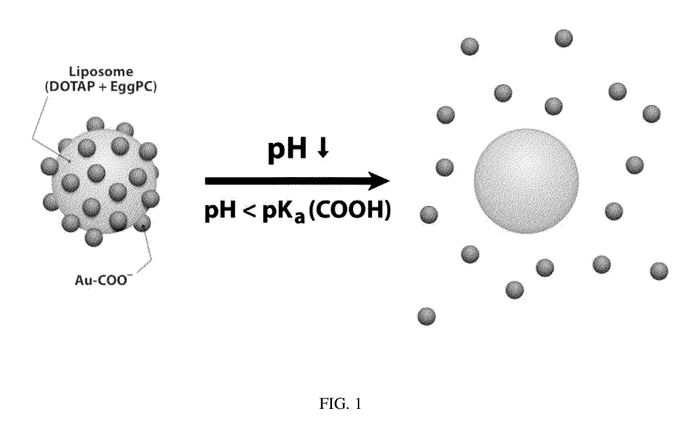

The application of carboxyl-modified biocompatible nanoparticles to mediate the fusion activity of phospholipid liposomes is illustrated in FIG. 1 using gold nanoparticles. With a pKa≈5, the carboxylic group is deprotonated at pH=7 resulting in negatively charged Au—COO— nanoparticles which bind to cationic liposomes through electrostatic attraction and thus stabilize the liposomes. When the environment pH drops to below 5, the carboxylic group is protonated. The resulting neutral Au—COOH nanoparticles detach from the liposome surface due to the lack of binding forces, thereby freeing the liposomes. Gold nanoparticles were selected for this example, and the Examples below, because of their fluorescence quenching properties that can be employed to indicate their binding and detaching process and extent when a small fraction of fluorescent dyes is doped into the liposome membranes. Moreover, gold is a biocompatible noble metal with antimicrobial activity against a wide type of bacteria. However, one of skill in the art would recognize alternative nanoparticles having similar attributes including those as defined above such as silver and synthetic biocompatible nanoparticles such as polystyrene.

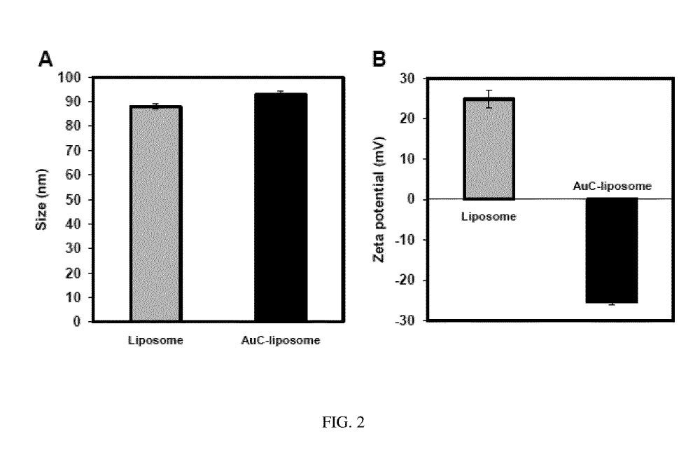

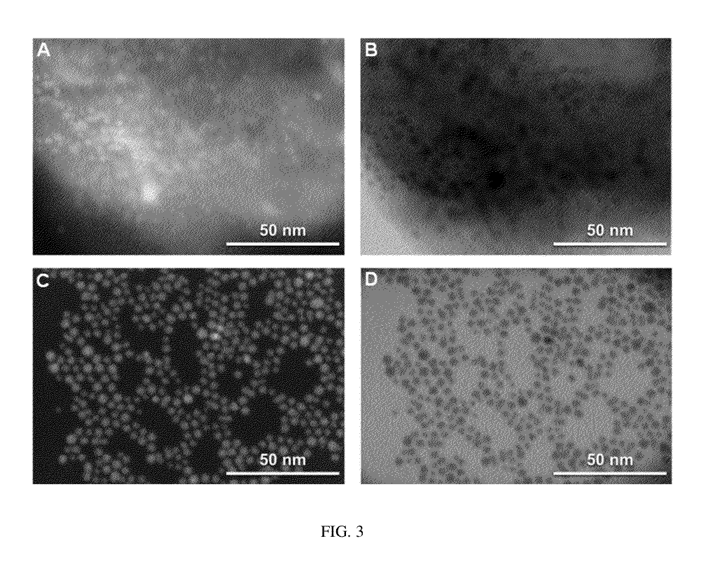

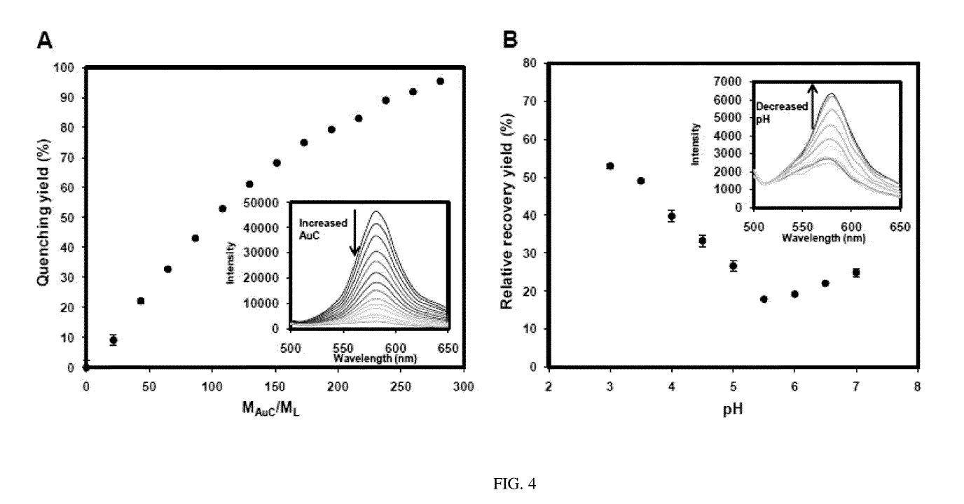

Cationic phospholipid liposomes consisting of 90 wt % hydrogenated L-α-Phosphatidylcholine (Egg PC) and 10 wt % 1,2-di-(9Z-octadecenoyl)-3-trimethylammonium-propane (DOTAP) were prepared through a well-known extrusion method (Mayer, L. D. et al. Vesicles of Variable Sizes Produced by a Rapid Extrusion Procedure. Biochim. Biophys. Acta 1986, 858, 161-168). Dynamic light scattering (DLS) measurements showed the size and surface zeta potential of the formed liposomes were 88.0±1.0 nm and 24.9±2.3 mV, respectively (FIG. 2). The positive zeta potential value indicates the incorporation of DOTAP to the liposome membrane. In a separate reaction, AuC nanoparticles were synthesized following a previously published protocol (Aryal, S. et al. Spectroscopic Identification of S—Au Interaction in Cysteine Capped Gold Nanoparticles. Spectrochim. Acta A 2006, 63, 160-163; Patil, V. et al. Role of Particle Size in Individual and Competitive Diffusion of Carboxylic Acid Derivatized Colloidal Gold Particles in Thermally Evaporated Fatty Amine Films. Langmuir 1999, 15, 8197-8206) resulting in AuC with a nearly uniform size of ˜4 nm measured by scanning transmission electron microscope (STEM) (FIG. 3) and a negative surface zeta potential of −25.6±4.2 mV determined by DLS. The synthesized cationic liposomes and AuC nanoparticles were mixed with a molar ratio of 1:200 under gentle bath sonication for 10 min to form AuC-liposome. The excess AuC in the solution was removed by 10 min centrifugation at 1.3×10⁴ rpm to ensure the subsequent particle size and surface zeta potential measurements were solely from the AuC-liposome but not from unbound AuC particles. DLS data showed that the size of the AuC-liposome was 92.9±1.3 nm and the surface zeta potential was −25.3±0.7 mV (FIG. 2). The measured AuC-liposome size was slightly larger than that of bare liposomes because of the adsorption of 4 nm AuC nanoparticles, while the change of zeta potential from 24.9 mV to −25.3 explicitly suggests the binding of negatively charged AuC to the positively charged liposomes. The morphology and structure of the AuC-liposome were further imaged by STEM. As shown in FIG. 3 AB, individual AuC particles were visible on the surface of liposomes after they were deposited on a TEM grid. Using the energy dispersive x-ray (EDX) spectrometer on the STEM, we were able to identify elementally that certain regions in FIG. 3 AB contained Au and other regions contained only elements found in the liposome such as carbon and phosphorus. The size of dehydrated liposomes was larger than the size of hydrated liposomes measured by DLS due to the collapse of liposomes from a 3 dimensional sphere to a 2 dimensional thin layer.

To further confirm the binding of AuC nanoparticles to the liposome surface, a fraction of fluorescently labeled lipid, 1,2-dimyristoyl-sn-glycero-3-phosphoethanolamine-N-lissamine rhodamine B sulfonyl (DMPE-RhB, Excitation/Emission=550 nm/590 nm), was doped into the liposome membranes. It was expected that AuC binding would quench the fluorescence dye underneath or nearby the AuC particles because of a fluorescence resonance energy transfer (FRET) mechanism. AuC nanoparticles were mixed with fluorescently labeled liposomes with a molar ratio (MAuC/ML) ranging from 0 to 280. Fluorescence emission intensity at 590 nm was recorded and quenching yield was calculated as following: quenching yield (%)=(1−I_{AuC}-L/IL)×100, in which I_{AuC}-L and IL represent the fluorescence intensity of RhB-labeled liposomes in the presence and absence of AuC nanoparticles, respectively. As shown in FIG. 4A, when M_{AuC}/M_{L} molar ratio increased, the quenching yield raised and reached 100% at M_{AuC}/M_{L}=280. Since the diameters of liposomes and AuC nanoparticles were about 88 nm and 4 nm, respectively, the surface coverage of AuC on liposome surface was about 14% at the M_{AuC}/M_{L} ratio of 280:1 if assuming all AuC attached to liposome surface. According to the FRET mechanism, the adsorbed AuC particles can effectively quench DMPE-RhB probes not only underneath the AuC but also within 2˜5 nm region surrounding the AuC particles. This will result in a near 100% theoretical quenching yield, which is consistent with what has been observed in FIG. 4A. Although more AuC particles might be able to adsorb onto the ˜86% unoccupied liposome surface, further studies demonstrated that the quenching yield remained as a plateau of 100% when more AuC were added into the solution above the fully quenching point of M_{Auc}/M_{L}=280. FIG. 4A inset showed the representative fluorescence emission spectra of the AuC-liposome in the range of 500-650 nm at different M_{AuC}/M_{L} ratios with an excitation wavelength of 470 nm. This excitation wavelength can effectively excite DMPE-RhB probe doped in liposome membranes while minimally interfering with the fluorescence emission spectra.

Without being bound to a particular theory, the present invention provides that when the environment pH value is reduced below the pKa value of carboxylic acid, the negatively charged Au—COO⁻ will be protonated to form neutral Au—COOH, which may detach from the cationic liposomes due to the elimination of electrostatic attraction. Subsequently, the detaching of AuC will induce a fluorescence recovery of the DMPE-RhB probes doped in the liposomes. To test this, AuC-liposome solution with a M_{AuC}/M_{L} ratio of 200 was used to study relative fluorescence recovery yield of DMPE-RhB at various pH values. The pH of the AuC-liposome solution was adjusted to desired values ranging from pH=7 to pH=3 using buffer solutions consisting of potassium hydrogen phthalate or potassium phosphate monobasic with a final salt concentration of 5 mM. Fluorescence emission intensity at 590 nm of the AuC-liposome solutions at various pH values was recorded. Considering the detached AuC nanoparticles suspending in the fluorescently labeled liposome solutions may quench the DMPE-RhB dyes as well through random collision, the relative recovery yield was used to describe the fluorescence recovery upon pH change. The fluorescence intensity of AuC-liposome at each pH point was normalized with that of liposomes mixing with the same amount of bare gold nanoparticles (AuB), which are neutral particles without carboxyl modification and characteristic of Au—COOH. The relative recovery yield was defined as following: Relative recovery yield (%)=I_{AuC}-L/I_{AuB}-L×100, in which I_{AuC}-L and I_{AuB}-L represent fluorescent intensity of AuC stabilized liposomes and mixture of liposomes and AuB at the same concentration as AuC-liposomes at various pH values. As shown in FIG. 4B, the relative recovery yield of DMPE-RhB labeled AuC-liposome slightly decreased from 23% to 18% when the pH value decreased from 7 to 5.5. Then it dramatically increased from 18% to about 55% when the pH value further decreased from 5.5 to 3. The slight decrease of the relative recovery yield from pH=7 to 5.5 indicates that more AuC particles adsorb onto the liposomes or stronger binding between AuC and liposomes occurs at pH=5.5 than at pH=7. This might be because cationic lipid DOTAP becomes more positively charged at lower pH resulting in stronger charge-charge attraction between AuC and the liposomes. While when the pH value was less than 5.5 within the range of 5.5-3, the protonation effect of AuC was more dominant than any other effects, which significantly weakened the electrostatic attraction. Therefore, AuC detached from the liposome surface leading to high fluorescence recovery. FIG. 4B inset showed the representative fluorescence emission spectra of the AuC-liposome in the range of 500-650 nm at different pH values ranging from 7 to 3 with an excitation wavelength of 470 nm. These fluorescence recovery results are consistent with the surface zeta potential measurements of the AuC-liposome at different pH values. The surface zeta potential of the AuC-liposome increased from −25.3±0.7 mV at pH=7 to +30.1±2.1 mV at pH=4, indicating the detachment of the AuC from the liposome surface at acidic pH. The surface zeta potential of the AuC-liposome at pH=4 is slightly higher than bare liposomes at pH=7, 24.9±2.3 mV (FIG. 2B), which may be because the cationic lipid DOTAP is more positively charged at acidic pH.

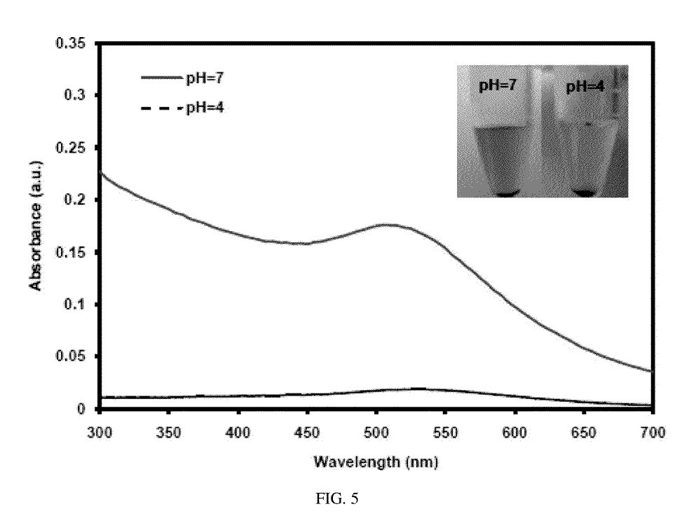

The binding of AuC to liposome surface at neutral pH and detaching at acidic pH were further examined by measuring UV-vis absorption of AuC-liposome at pH=7 and pH=4, respectively, after the removal of unbound AuC via proper centrifugation. Here HCl was used to adjust the pH of the AuC-liposome solutions instead of using buffer solutions because some UV absorption of the buffer was detected. After incubating the AuC-stabilized cationic liposomes (not fluorescently labeled) with HCl for 10 min at pH=7 and pH=4, respectively, the AuC-liposome solutions were centrifuged to precipitate unbound AuC nanoparticles. The UV-vis absorption spectra of the resulted supernatants were then recorded in the range of 300 nm to 700 nm as shown in FIG. 5. At pH=7, UV absorption spectrum of AuC was clearly detected but not at pH=4. The observed UV absorption spectra were consistent with the color difference of the supernatant as shown in FIG. 5 inset. At pH=7, a small amount of particle precipitates was observed while the color of the supernatant remained as red, characteristic of gold nanoparticles. In contrast at pH=4, a large amount of particle precipitates appeared and the color of the supernatant became clear. This clear supernatant was then subjected to measuring the size and surface zeta potential using DLS with results similar as bare liposomes. These data suggest that when the pH value (e.g. pH=7) was higher than the pKa (˜5) of carboxylic acid, AuC were in deprotonated form (Au—COO⁻) and thus strongly bound to cationic liposomes. So they could not be separated from liposomes by centrifugal force. However, when the pH value (e.g. pH=4) was less than the pKa value, AuC were protonated to Au—COOH form which no longer adsorbed on the liposomes. The unbound Au—COOH particles were readily separated from the solution by centrifugation.

After having demonstrated the binding and detaching of AuC nanoparticles from cationic liposomes upon environment acidity changes, the controllable fusion activity of the liposomes mediated by the AuC nanoparticles was examined. To this end, anionic liposomes consisting of Egg PC and lauric acid (LA) were preprared, which were mixed with AuC-stabilized cationic liposomes at different pH values. It was expected that bare cationic liposomes would bind to and fuse with anionic liposomes intimately after the AuC were protonated and detached from the cationic liposomes. To monitor the fusion process and the fusion extent, the anionic liposomes were pre-labeled with a FRET pair of chromophores, and the change in FRET signal was measured upon mixing the FRET-labeled anionic liposomes with AuC-stabilized cationic liposomes at pH=7 and pH=4, respectively. FRET is a widely used technique that precisely measures the distance of two subjects at the molecular level based on an energy transfer mechanism of two chromophores. When the two chromophores are in close proximity (<10 nm), excited donor can transfer energy to the acceptor through a nonradiative long-range dipole-dipole coupling mechanism. Here we incorporated a fluorescence donor (C6NBD: excitation/emission=470 nm/520 nm) and a fluorescence acceptor (DMPE-RhB: excitation/emission=550 nm/590 nm) into the lipid membranes of anionic liposomes. By controlling the molar ratio between the donor and the acceptor, the fluorescent anionic liposomes in which the fluorescence emission from the donor was completely quenched by the acceptor. If the anionic liposomes fuse with the cationic liposomes, the spread of the donor and acceptor chromophores within the cationic liposomes will alleviate or eliminate the FRET efficiency, resulting in fluorescence recovery of the donor.

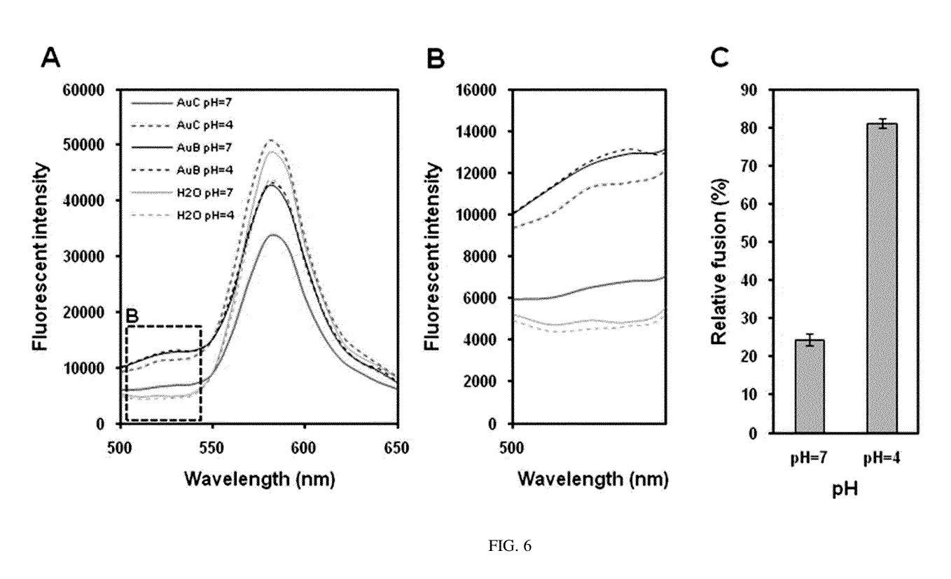

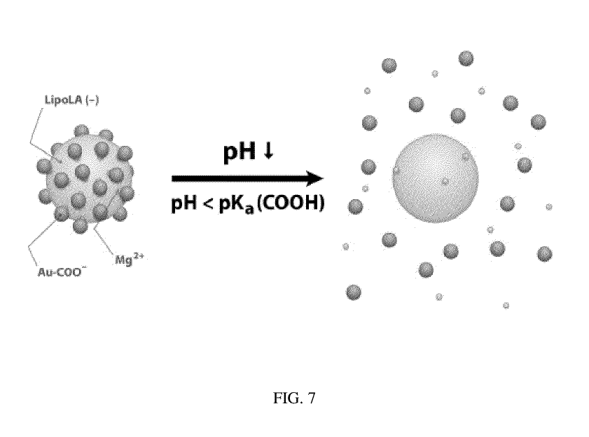

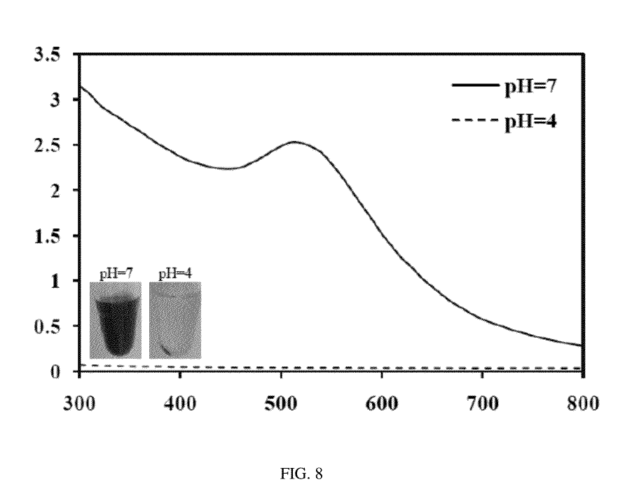

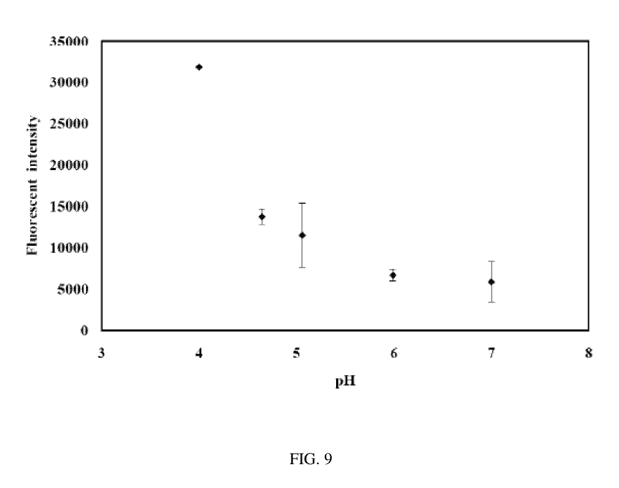

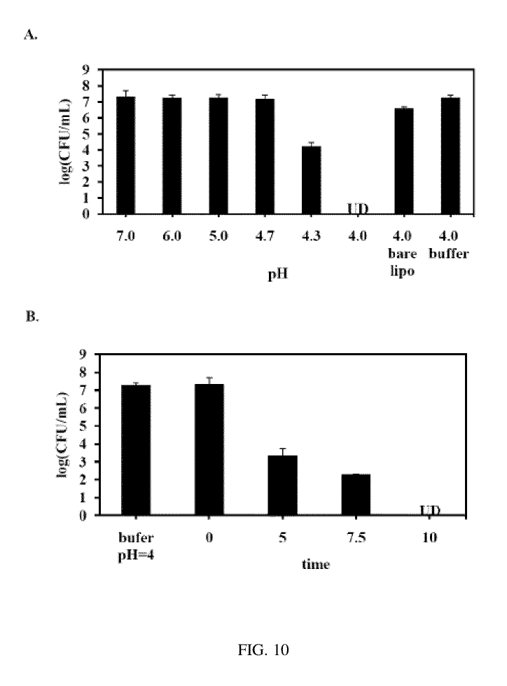

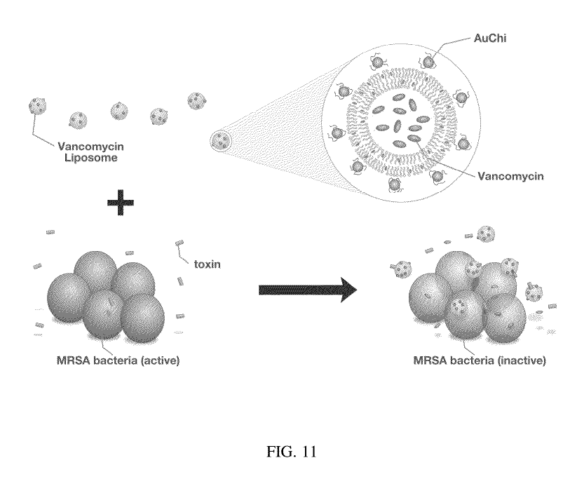

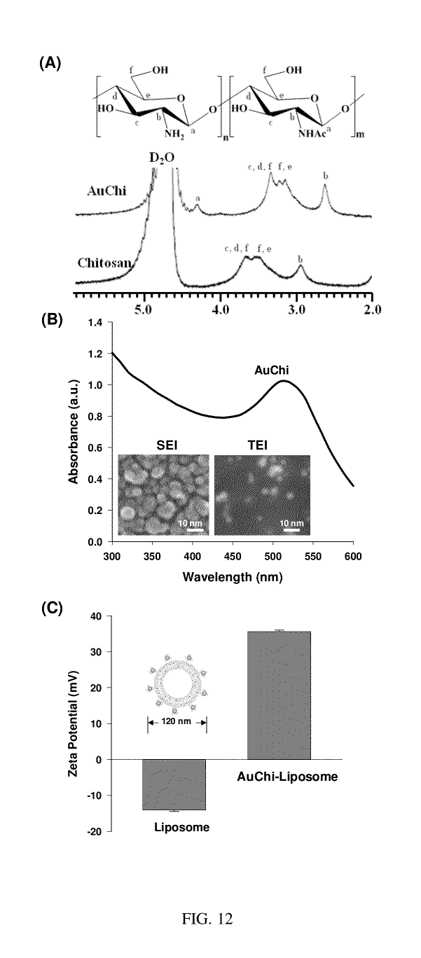

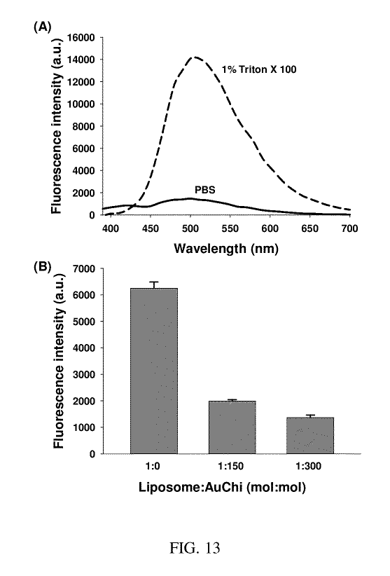

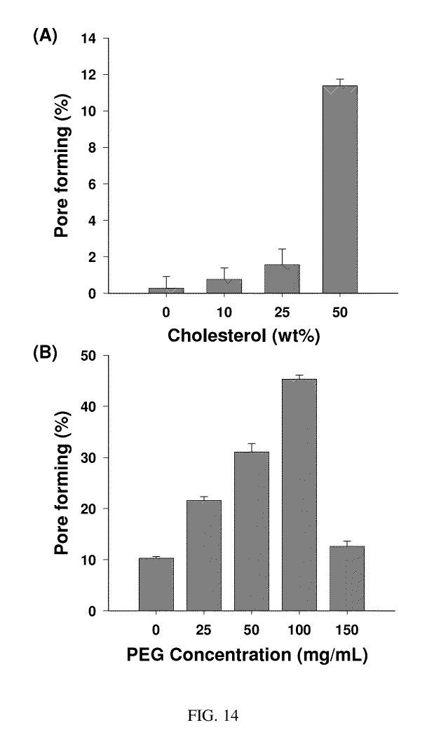

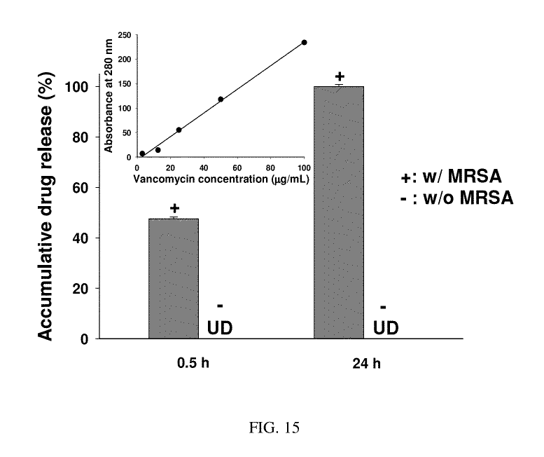

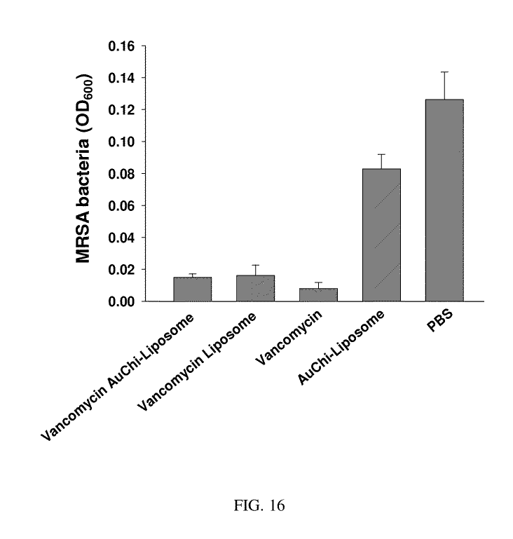

For this fusion example, AuC-stabilized cationic liposomes (M_{AuC}/M_{L}=200) were first adjusted to pH=7 and pH=4, respectively, using buffer solutions. The resulting unbound AuC nanoparticles were removed from the solutions via 10 min centrifugation at 1.3×10⁴ rpm in order to eliminate fluorescence quenching effect of free AuC in the solutions through random collision. Subsequently, the cationic liposomes were mixed with the FRET-labeled anionic liposomes at a molar ratio of 7:1. The mixtures were then excited at the wavelength of 470 nm and fluorescence emission spectra in the range of 500-650 were recorded as shown in FIG. 4A. Since the fluorescent receptor DMPE-RhB was also excited at the 470 nm resulting in a dominant emission peak at 590 nm, we zoomed in to the 500-540 nm emission window which was predominantly from the C6NBD (FIG. 6B). We found that significant fluorescence recovery of C6NBD occurred at pH=4 as compared to at pH=7. The best explanation is that at pH=7 Au—COO— nanoparticles strongly bind to the cationic liposomes and prevent them from fusion with anionic liposomes. However, at pH=4 the protonated Au—COOH nanoparticles detach from the cationic liposomes, resulting in bare cationic liposomes that effectively fuse with the anionic liposomes. To rule out the possibility that pH adjustment affects the FRET efficiency within the anionic liposomes, FRET-labeled anionic liposomes adjusted to the corresponding pH values and concentrations without mixing with cationic liposomes were applied as negative controls. When the control samples were excited at 470 nm, no considerable fluorescence emission difference at 530 nm was detected at pH=7 and pH=4. Additionally, AuB nanoparticles (neutral and no carboxyl modification) were used as positive controls. Strong fluorescence emission of C6NBD at 530 nm appeared at both pH=7 and pH=4, indicating that AuB do not bind tightly to the cationic liposomes to prevent them from fusion with the anionic liposomes at both neutral and acidic pH values. FIG. 6C highlighted the relative fusion efficiency of AuC-cationic liposomes with anionic liposomes over AuB-cationic liposomes with anionic liposomes, taking anionic liposomes alone at the corresponding pH values and concentrations as background. The relative fusion ability at different pH values were calculated as following: Relative fusion (%)=(I_{530,AuC}−I_{530,H2O})/(I_{530,AuB}−I_{530,H2O})×100, in which I_{530,AuC} represents fluorescence emission intensity at 530 nm of the AuC-cationic liposomes mixing with the anionic liosomes I_{1530,AuB} represents fluorescence emission intensity at 530 nm of the AuB-cationic liposomes mixing with the anionic liosomes; I_{530,H2O} represents fluorescence emission intensity at 530 nm of the anionic liposomes alone. As shown in FIG. 6C, the relative fusion yield of AuC-cationic liposomes was 24.4±1.6 at pH=7 and 81.1±1.2 at pH=4, indicating the feasibility of using AuC to mediate the fusion activity of liposomes.

Having successfully synthesized carboxyl-modified AuNP (denoted as AuC, diameter ˜4 nm) and demonstrated that they can bind to and detach from cationic phospholipid liposomes at different pH values, AuC was used to control the fusion activity of liposomal lauric acid (LipoLA) and to enable skin-sensitive lauric acid drug delivery. With a pKa≈5, the carboxylic group is deprotonated at pH=7 resulting in negatively charged Au—COO⁻, which can bind to LipoLA in the presence of divalent ions such as magnesium (Mg²⁺) through electrostatic attraction and thus stabilize the liposomes. When the environment pH drops to below 5, the carboxylic group is protonated. The resulting neutral Au—COOH detach from the LipoLA surface due to the lack of binding forces, thereby freeing the liposomes.

AuC—Mg-lipoLA, lipoLA composing of eggPC and LA (3:2 weight ratio) were prepared through the well-known extrusion method (see above). The non-encapsulated LA was separated from the liposomes on a column of Sephadex G75. In a separate reaction, AuC nanoparticles were synthesized following a previously published protocol (see above). AuB were functionalized with carboxyl groups by overnight incubation with MPA (4×10⁻⁴M). The resulting AuC were washed 3 times by an Amicon Ultra-4 centrifugal filter with a molecular weight cut-off of 10 kDa (Millipore, Billerica, Mass.). The resulting lipoLA, MgSO₄ and AuC were mixied (1:2000:200 molar ratio) under gentle bath sonication for 10 min in order to yield AuC—Mg-LipoLA.

The reactivation of the fusion ability of LipoLA upon the detaching of AuC was tested for P. acnes. RhB—AuC—Mg-LipoLA (140 μg/mL of initial lipid concentration) were mixed with P. acnes of 7.93×10⁸ CFU/mL, and pH of the solution were adjusted from 4 to 7 by buffer solution. After 15 min incubation at room temperature, samples were centrifuged at 13,200 rpm for 5 min to remove the excess amount of RhB—AuC—Mg-LipoLA and were resuspended in PBS. To determine the antimicrobial activity of AuC—Mg-LipoLA against P. acnes, AuC—Mg-LipoLA with pH ranges from 4.0 to 7.0, adjusted by buffer solution, were incubated with P. acnes (5×10⁷ CFU/mL) at 37° C. for the desired incubation time under anaerobic condition. (FIG. 9) The results showed that at pH 4.0, AuC—Mg-LipoLA completely killed P. acnes. Incubation of P. acnes with empty liposome solution (without LA) and buffer at pH 4 served as negative controls.

### Triggered Cargo Release Via Liposome Pore Formation

The present invention also provides a passive targeting cargo delivery platform in which pore forming toxins, among other triggers, are utilized to trigger cargo release from biocompatible nanoparticle-stabilized liposomes at a target dermal site. In particular, a passive targeting antimicrobial drug delivery platform is provided in which bacterial toxins are utilized to trigger antibiotic release from gold nanoparticle-stabilized liposomes for inhibiting the growth of the toxin-secreting bacteria.

In one example, the liposome composition and the coverage of chitosan modified gold nanoparticles on the liposome surface were optimized so that the liposome fusion activity and undesirable drug leakage were prohibited at normal storage condition, while the liposomes were still susceptible to pore-forming toxins. Once incubated with toxins, the liposomes became leaky and the cargo, in this case encapsulated antibiotic payloads, were rapidly released through the toxin-formed pores. It was further demonstrated that in the presence of toxin-secreting bacteria, 100% of the cargo, in this case encapsulated antibiotics, were released from gold nanoparticle-stabilized liposomes and bacterial growth was effectively inhibited by the released antibiotics in 24 h. This antimicrobial drug delivery approach provides a new paradigm for the treatment of bacterial infections by specifically releasing drugs at the infectious sites while minimizing off-target effects. While vancomycin was used as an anti-methicillin-resistant Staphylococcus aureus (MRSA) antibiotic in this study, this technique can be generalized to selectively deliver cargo for the treatment of various conditions caused by bacteria and other organisms that secrete pore-forming toxins. In addition, this technique can be generalized to selectively deliver cargo to target dermal sites for the treatment of other conditions. In yet another example, this system can be modified to deliver chemotherapeutic agents to cancerous dermal lesions, such as melanoma. Doxorubicin is an example of cargo that can be selectively delivered to melanoma lesions, and by triggering the release of the doxorubicin via application of a synthetic pore forming toxin, such as Triton-X100®, at the site of the cancerous lesion.

As provided in the Examples below, using MRSA as a model bacterium and vancomycin as a model anti-MRSA antibiotic, the synthesized gold nanoparticle-stabilized liposomes were demonstrated to completely release the encapsulated vacomycin within 24 h in the presence of MRSA bacteria and lead to inhibition of MRSA growth as effective as an equal amount of vancomycin loaded liposomes (without nanoparticle stabilizers) and free vancomycin. This bacterial toxin enabled drug release from nanoparticle-stabilized liposomes provides a new, safe and effective approach for the treatment of bacterial infections. This technique can be broadly applied to treat a variety of infections caused by bacteria that secrete pore forming toxins. Those of skill in the art will readily recognize such compounds, including those as defined above.

The present invention provides that the nanoparticle stabilized liposomes are as effective as an equal amount of vancomycin loaded liposomes (without nanoparticle stabilizers) and free vancomycin, which demonstrates the potential of nanoparticle stabilized liposomes formulations to improve drug potency and overcome drug resistance. Second, controlled drug release from liposome-based formulations has long been considered challenging. The Examples herein takes the advantage of pore-forming property of the toxin and develops biomimetic strategy to release encapsulated drugs in a controlled fashion. In various aspects, the amount of the drug released is self-regulated and correlates to the bacterial viability. This correlation is significant because it can effectively minimize drug systemic exposure and off-target delivery, and improve delivered drug potency.

Instead of using targeting ligands to actively target the drug carriers to the bacteria of interest, the present invention takes advantage of pore forming molecules, such as toxins secreted by target bacteria, and uses them to trigger the release of cargo to target dermal sites. In various aspects, the use of such nanoparticle stabilized liposomes can kill target bacteria. Using this approach, prior to contact with the target bacteria, drugs are protected inside the liposomes and are not released, thereby eliminating adverse side effects due to premature drug leakage or non-specific drug release. As a proof-of-concept, it is demonstrated in the Examples that bacterial toxins can be utilized to trigger antibiotic release from gold nanoparticle-stabilized phospholipid liposomes and the released antibiotics can subsequently inhibit the growth of, in a non-limiting example, Staphylococcus aureus (S. aureus) bacteria that secrete the toxins.

As mentioned above, there are a variety of molecules that possess pore-forming activity, including bacterial toxins, animal toxins, immune proteins, and synthetic compounds such as Triton X-100®. Alpha hemolysin, also named α-toxin, is one of the common toxins secreted by S. aureus bacteria as a water-soluble protein monomer with a molecular weight of 34 kDa. This protein can spontaneously incorporate into lipid membranes and self oligomerize to form a heptameric structure with a central pore. The pore size is about 2 nm that allows small molecules up to about 3 KDa to passively diffuse through the membranes. In nature, S. aureus bacteria secrete α-toxin that can bind to the outer membranes of susceptible cells. Upon binding, rapid pore forming facilitates uncontrolled permeation of water, ions, and small molecules, rapid discharge of vital molecules such as ATP, dissipation of the membrane potential and ionic gradients, and irreversible osmotic swelling leading to the cell lysis. Considering the tremendous availability of pore forming toxins at bacterial infection sites and their pore forming activities, the present invention provides that these invasive molecules can be utilized to selectively release cargo, including antimicrobials, from liposomes that are stabilized by small biocompatible, including gold, nanoparticles to avoid undesirable membrane-membrane fusion and drug leakage. This strategy allows selective release of drugs at the infectious sites to kill toxin-secreting bacteria while not producing any toxic side effects on healthy tissues.

The invention further provides synthesis of a novel liposome formulation stabilized by chitosan-modified gold nanoparticles (AuChi) to differentially release cargo, and in a non-limiting example vancomycin, to inhibit the growth of, in yet another non-limiting example, S. aureus bacteria for topical treatment of skin bacterial infections. FIG. 11 illustrates the working principle of toxin-triggered antibiotic release from gold nanoparticle-stabilized liposomes for the treatment of the bacteria that secrete the toxins. The cationic AuChi bind to the negatively charged liposome surfaces through electrostatic attraction and thus stabilize liposomes against fusion with one another and avoid undesirable antibiotic leakage. When the stabilized liposomes are in the vicinity of S. aureus bacteria, the bacterium-secreted toxins will insert into liposome membrane and create pores, through which the encapsulated antibiotic is released. The released vancomycin, as staying in close to the bacteria, will then exert its antimicrobial activity rapidly and locally.

### Topical Administration

Liposomes made by the processes of the present invention may serve as delivery vehicles for medicaments for treating dermal conditions or as intermediates for the synthesis of compositions that are pharmaceutically active agents for treating dermal conditions, including, but not limited to, MRSA infection and P. acnes infection. A non-limiting example of dermal administration includes U.S. Pat. Nos. 5,830,877, 6,245,347, 7,754,240, as such parts relevant to dermal formulations and dermal administration routes are incorporated herein by reference.

The medicaments delivered by the processes of the present invention and where appropriate, their pharmaceutically acceptable salts, may be topically administered dermally and transdermally. Amounts of the medicaments delivered correlate to the trigger that activates the release of the medicament. This can include administration of dosages administered ranging from about 0.01 mg up to about 1500 mg per day, although variations may occur depending upon the condition of the persons being treated and their individual responses to said medicament, as well as on the type of pharmaceutical formulation chosen and the time period and interval during which such administration is carried out. In some instances, dosage levels below the lower limit of the aforesaid range may be more than adequate, while in other cases still larger doses may be administered without causing any harmful side effects.

The medicaments delivered by the processes of the present invention and where appropriate, their pharmaceutically acceptable salts, may be administered alone or in combination with pharmaceutically acceptable carriers or diluents by any of the routes previously indicated. More particularly, the compounds may be administered in a wide variety of different dosage forms, e.g., they may be combined with various pharmaceutically acceptable inert carriers in the form of transdermal patches, powders, sprays, creams, salves, jellies, gels, pastes, lotions, ointments, aqueous suspensions, and the like. Such carriers include solid diluents or fillers, sterile aqueous media and various non-toxic organic solvents.

As various changes could be made in the above compounds, products and methods without departing from the scope of the invention, it is intended that all matter contained in the above description and in the examples given below, shall be interpreted as illustrative and not in a limiting sense.

### EXAMPLES

Aspects of the present teachings may be further understood in light of the following examples, which should not be construed as limiting the scope of the present teachings in any way.

### Example 1

### Preparation of Carboxyl-Modified Gold Nanoparticles

Preparation of Carboxyl-Modified Gold Nanoparticles (AuC).

AuC were prepared by sodium borohydride reduction method described in full details elsewhere (Aryal, S. et al. Spectroscopic Identification of S—Au Interaction in Cysteine Capped Gold Nanoparticles. Spectrochim. Acta A 2006, 63, 160-163; Patil, V. et al. Role of Particle Size in Individual and Competitive Diffusion of Carboxylic Acid Derivatized Colloidal Gold Particles in Thermally Evaporated Fatty Amine Films. Langmuir 1999, 15, 8197-8206). Briefly, aqueous solution of HAuCl₄ (10⁻⁴M, 50 mL) was reduced by 0.005 g of NaBH₄ at ice cold temperature, resulting in the formation of bare gold nanoparticles (AuB). AuB were functionalized with carboxyl groups by overnight incubation with MPA (mercaptopropionic acid, 4×10⁻⁴M). The resulting AuC were washed 3 times by an Amicon Ultra-4 centrifugal filter with a molecular weight cut-off of 10 kDa (Millipore, Billerica, Mass.) and suspended in aqueous solution at pH=6.8.

Preparation and Characterization of Liposomes and AuC-Liposomes.

Cationic liposomes consisting of Egg PC (zwitterionic phosphalipid) and DOTAP (cationic phospholipid) were prepared through the well-known extrusion method (Mayer, L. D. et al. Vesicles of Variable Sizes Produced by a Rapid Extrusion Procedure. Biochim. Biophys. Acta 1986, 858, 161-168). Briefly, 1.5 mg of Egg PC and DOTAP mixture (weight ratio=9:1) were dissolved in 1 mL of chloroform. The solvent was evaporated by blowing argon gas over it for 15 min. Then the dried lipid films were hydrated with 3 mL of deionized water, followed by vortexing for 1 min and sonicating for 3 min in a bath sonicator (Fisher Scientific FS30D) to produce multilamellar vesicles (MLVs). A Ti-probe (Branson 450 sonifier) was used to sonicate the MLVs for 1-2 minutes at 20 W to produce unilamellar vesicles. To form narrowly distributed small unilamellar vesicels (SUVs), the solution was extruded through a 100 nm pore-sized polycarbonate membrane for 11 times. AuC-stabilized liposomes (AuC-liposomes) were prepared by mixing liposomes and AuC nanoparticles at desired molar ratios under gentle bath sonication for 10 min.

The hydrodynamic size and surface zeta potential of the prepared liposomes and AuC-liposomes were assessed by using the Malvern Zetasizer ZS (Malvern Instruments, UK). The mean diameter and zeta potential were determined through dynamic light scattering (DLS) and electrophoretic mobility measurements, respectively. All characterization measurements were repeated three times at 25° C. The morphology and structure of the AuC-liposome were characterized by a Hitachi HD2000 scanning transmission electron microscope (STEM) equipped with a cold cathode field emission electron source and a turbo-pumped main chamber. Samples for STEM characterization were prepared by dispersing a solution containing the AuC-liposome onto the surface of a carbon film coated Cu grid. The samples were air-dried, and then coated with a thin amorphous carbon film by evaporation. All images were recorded in the STEM as scanned beam images, using the secondary electron signal, which provides surface topology detail, the direct transmitted electron beam (unscattered electrons) or the diffracted transmission electrons collected on an annular dark field detector.

Fluorescence Quenching and Recovery Studies.

DMPE-RhB labeled liposomes were prepared by mixing 0.5 mol % DMPE-RhB with Egg PC and DOTAP prior to liposome preparation. To monitor the quenching effect of AuC on the fluorescently labeled liposomes, AuC were mixed with the liposomes at desired molar ratios (MAuC/ML) ranging from 0 to 280, followed by 10 min sonication. The fluorescence emission spectra of DMPE-RhB in the range of 500-650 nm were measured by using a fluorescent spectrophotometer (Infinite M200, TECAN, Switzerland) at an excitation wavelength of 470 nm. The emission peak at 590 nm was selected to quantify the fluorescence quenching yield.

To study fluorescence recovery yield of DMPE-RhB labeled AuC-liposome at different pH values, the AuC-liposome solution with a MAuC/ML=200 was selected. The DMPE-RhB labeled AuC-liposome were adjusted to desired pH values using proper buffer solutions with target pH values (potassium hydrogen phthalate buffer for pH=3-5, and potassium phosphate monobasic buffer for pH=5.5-7). The actual pH value of each AuC-liposome solution was measured by an Orion 3-star plus portable pH meter. The salt concentration of each AuC-liposome solution after pH adjustment was 5 mM. The fluorescence emission spectra of DMPE-RhB were measured as previously described. The mixtures of fluorescently labeled liposome and bare gold nanoparticles (AuB, no carboxyl modification) at the same molar ratios were used as positive controls.

UV-Vis Absorption Spectra of AuC-Liposomes at pH=7 and 4.

AuC-liposome were prepared following the protocol described above. To adjust the pH value of the AuC-liposome solution to pH=4, 0.1 M HCl was used because it did not induce any undesirable UV absorption background. Unbound AuC were removed from the solution by centrifugation at 1.3×10⁴ rpm for 10 min. Absorption spectra in the range of 300 nm to 700 nm were recorded by a spectrophotometer. To exclude possible UV absorption from the cationic liposomes and background, free liposomes (without AuC addition) at the same concentration and pH value as the AuC-liposome were measured, whose signal was subtracted from the measured AuC-liposome UV absorption spectra. All measurements were repeated three times.

AuC-Liposome Fusion Studies.

To investigate the fusion activity of AuC-liposome against other liposomes or target cells at different pH values, negatively charged liposomes consisting of Egg PC and lauric acid (weight ratio=9:1) were synthesized by extrusion method as described above to mimic negatively charged cells. These anionic liposomes were labeled with a fluorescence resonance energy transfer (FRET) pair of chromophores, a florescent donor (C6NBD, 0.1 mol %) and a fluorescent quencher (DMPE-RhB, 0.5 mol %). AuC-cationic liposomes (MAuC/ML=200) solutions were prepared and adjusted to pH=7 and pH=4, respectively. Unbound AuC nanoparticles were removed by centrifugation at 1.3×10⁴ rpm for 10 min. The supernatants of the AuC-cationic liposomes were mixed with FRET-labeled anionic liposomes with a molar ratio of 7:1. Consequently, fluorescence emission spectra at the range of 500-650 nm were obtained by exciting the samples at 470 nm using a fluorescent spectrophotometer. AuB-cationic liposome mixtures at the corresponding molar ratios and pH values were used as positive controls. The FRET-labeled anionic liposomes alone (without the addition of cationic liposomes) at the corresponding concentrations and pH values were used as negative controls. All measurements were carried out at 25° C. and repeated three times.

Synthesis of Acid-Responsive AuC—Mg-lipoLA.

To prepare AuC—Mg-lipoLA, lipoLA composing of eggPC and LA (3:2 weight ratio) were firstly prepared by the extrution method above. The non-encapsulated LA was separated from the liposomes on a column of Sephadex G75. AuC were prepared by sodium borohydride reduction method. Aqueous solution of HAuCl₄ (10⁻⁴M, 50 mL) was reduced by 0.005 g of NaBH₄ at ice cold temperature, resulting in the formation of bare gold nanoparticles (AuB). AuB were functionalized with carboxyl groups by overnight incubation with MPA (4×10⁻⁴ M). The resulting AuC were washed 3 times by an Amicon Ultra-4 centrifugal filter with a molecular weight cut-off of 10 kDa (Millipore, Billerica, Mass.). The resulting lipoLA, MgSO₄ and AuC were mixied (1:2000:200 molar ratio) under gentle bath sonication for 10 min in order to yield AuC—Mg-LipoLA. To adjust the pH value of the AuC—Mg-lipoLA solution to pH=4, 0.1 M HCl was used because it did not induce any undesirable UV absorption background. Unbound AuC were removed from the solution by centrifugation at 1.3×10⁴ rpm for 10 min. Absorption spectra in the range of 300 nm to 800 nm were recorded by a spectrophotometer. To exclude possible UV absorption from the cationic liposomes and background, free liposomes (without AuC addition) at the same concentration and pH value as the AuC-liposome were measured, whose signal was subtracted from the measured AuC-liposome UV absorption spectra. All measurements were repeated three times.

Fusion Between Auc—Mg-lipoLA and P. acnes Bacteria.

RhB—AuC—Mg-LipoLA (140 μg/mL of initial lipid concentration) were mixed with P. acnes of 7.93×10⁸ CFU/mL, and pH of the solution were adjusted from 4 to 7 by buffer solution. After 15 min incubation at room temperature, samples were centrifuged at 13,200 rpm for 5 min to remove the excess amount of RhB—AuC—Mg-LipoLA and were resuspended in PBS. Consequently, emission intensity at 580 nm was obtained by exciting the samples at 550 nm using a fluorescent spectrophotometer (Infinite M200, TECAN, Switzerland).

Antimicrobial Activity of Auc—Mg-lipoLA Against P. acnes.

To determine the antimicrobial activity of AuC—Mg-LipoLA against P. acnes, AuC—Mg-LipoLA with pH ranges from 4.0 to 7.0, adjusted by buffer solution, were incubated with P. acnes (5×10⁷ CFU/mL) at 37° C. for the desired incubation time under anaerobic condition. The samples were diluted 1:10 to 1:106 in PBS, and 5 μL of dilutions was spotted on reinforced clostridial medium agar plates. Agar plates were incubated at 37° C. under anaerobic condition for 3 days, and CFU (colony forming units) of P. acnes was quantified. Buffer solution and empty liposomes (without LA, pH 4.0) were used as negative controls.

### Example 2

### MRSA Therapy Via Liposome Pore Formation

### Experimental Methods

Materials.

Hydrogenated L-α-phosphatidylcholine (Egg PC) and cholesterol were purchased from Avanti Polar Lipids, Inc. (Alabaster, Ala.). Sephadex G-75 was purchased from Fisher Scientific (Pittsburgh, Pa.). 8-aminonaphthalene-1,3,6-trisulfonic acid disodium salt (ANTS) and p-xylene-bis-pyridinium bromide (DPX) were obtained from Invitrogen (Carlsbad, Calif.). Poly(ethylene glycol) methyl (Mn=2000 Da) and Triptic Soy Broth (TSB) were purchased from Sigma Aldrich (St Louis, Mo.). Hydrogen tetrachloroaurate (HAuCl₄) and sodium borohydride (NaBH₄) were from ACROS Organics (Geel, Belgium). Chitosan-50 was purchased from Wako Pure Chemical Industries, Ltd. (Osaka, Japan).

Preparation and Characterization of AuChi and AuChi-Liposome.

Chitosan-modified gold nanoparticles (AuChi) were prepared by a sodium borohydride reduction technique (Pornpattananangkul, D. et al. ACS Nano 2010, 4, 1935-1942; Aryal, S. et al. Spectrochim. Acta A 2006, 63, 160-163). Briefly, aqueous solution of HAuCl₄ (10-4M, 50 mL) was reduced by 0.005 g of NaBH₄ at ice cold temperature to prepare bare gold nanoparticles. The acquired bare gold nanoparticles were then incubated overnight with 0.1% w/v chitosan that was pre-dissolved in 0.1 M acetic acid. The resulting AuChi were purified 3 times by an Amicon Ultra-4 centrifugal filter with a molecular weight cut-off of 10 kDa (Millipore, Billerica, Mass.).

Liopsomes were prepared following a previously described extrusion method. Briefly, 9 mg of lipid components were dissolved in 1 mL chloroform, and then the organic solvent was evaporated by blowing argon gas over the solution for 15 minutes to form a dried lipid film. The lipid film was rehydrated with 3 mL of deionized water with ANTS/DPX dyes or vancomycin, followed by vortexing for 1 min and sonicating for 3 min in a bath sonicator (Fisher Scientific FS30D, Pittsburgh, Pa.) to produce multilamellar vesicles (MLVs). Then the obtained MLVs were sonicated for 1-2 min at 20 W by a Ti-probe (Branson 450 sonifier, Danbury, Conn.) to produce unilamellar vesicles. The solution was extruded through a 100 nm pore-sized polycarbonate membrane for 11 times to form narrowly distributed small unilamellar vesicels (SUVs). The liposomes were purified by gel filtration with a Sephadex G-75 column equilibrated with water or isotonic PBS solution to remove unencapsulated dyes or drugs. To prepare AuChi-stabilized liposomes (AuChi-Liposome), the pH of both AuChi and liposome solutions was adjusted to 6.5 using HCl. Then the liposomes and AuChi at desired molar ratio were mixed together, followed by 10 min bath sonication, to prepare AuChi-Liposome.

UV-Vis absorbance spectrum of AuChi from 300 to 600 nm was recorded by a spectrophotometer (Infinite M200, TECAN, Männedorf, Switzerland). The morphology of the AuChi was characterized by a scanning transmission electron microscope (STEM) equipped with a cold cathode field emission electron source and a turbo-pumped main chamber (Hitachi HD2000, Tokyo, Japan). The STEM was operated at 200 keV accelerating voltage and 20 mA current, and images were recorded in both secondary electron mode and transmitted electron mode. Elemental analysis was performed with an EDAX energy dispersive x-ray spectrometer (EDS). Malvern Zetasizer ZS (Malvern Instruments, Worcestershire, UK) was used to measure the hydrodynamic size and surface zeta potential of the prepared AuChi, liposome, and AuChi-Liposome. The mean liposome diameter and surface zeta potential were determined by dynamic light scattering (DLS) and electrophoretic mobility measurements respectively. All characterization measurements were repeated three times at 25° C.

AuChi-Liposome Stability.

Liposomes, loaded with 12.5 mM of ANTS and 45 mM of DPX, were mixed with AuChi at different molar ratios (1:0, 1:150, or 1:300). The obtained AuChi-Liposome were incubated with bare liposomes, which were neither loaded with dyes nor stabilized by AuChi, at a molar ratio of 1:4 for 1 h at room temperature. The samples were then filtered through a Microcon YM-100 centrifugal filter with a molecular weight cut-off of 100 kDa (Millipore, Billerica, Mass.) for 20 min at 13.2×10³ rpm. The amount of ANTS in the filtrate was measured for its fluorescence emission intensity at 510 nm using a fluorescent spectrophotometer (Infinite M200, TECAN, Mannedorf, Switzerland) with an excitation wavelength of 360 nm.

Pore Forming Assay.

To study the pore forming activity of α-toxin against liposomes, 12.5 mM of ANTS and 45 mM of DPX were co-encapsulated into the liposomes, at which the fluorescence of ANTS was maximally quenched by DPX. The resulting liposomes (600 μg/mL) were then incubated with α-toxin (20 μg/mL) for 1 hr at room temperature. Once the pore forms, the encapsulated dyes will leach out of the liposomes, resulting in a florescence recovery of ANTS. After incubation, the fluorescence emission intensity of ANTS at 510 nm was measured by using a fluorescent spectrophotometer with an excitation at 360 nm. To obtain maximal dye leakage, Triton X-100 (1% v/v) was used as a positive control to completely lyse the liposomes. ANTS/DPX loaded liposomes at the corresponding concentrations in the absence of α-toxin served as a negative control and experimental background. To determine the optimal liposome formulation, liposomes composed of Egg PC and cholesterol (0, 10 wt %, 25 wt %, 50 wt %) were prepared and loaded with ANTS/DPX dyes to test their pore forming property, respectively. The effect of PEG on liposome pore forming property was assessed by adding PEG into the liposome solutions at various PEG concentrations: 1, 25, 50, 100, or 150 mg/mL.

Toxin-Triggered Vancomycin Release.

Vancomycin (10 mg/mL) loaded liposomes were stabilized by AuChi (Vancomycin AuChi-Liposome). To measure the drug loading yield of Vancomycin-Liposome and Vancomycin AuChi-Liposome, 1 mL of the liposome solution was vacuum dried for 2 h to remove all the liquid, the pallet was then reconstituted with 500 L water. The obtained suspension was centrifuged at 5000 rpm for 5 min and the supernatant was collected for reversed phase high performance liquid chromatography (HPLC) using Agilent 1100 series (Santa Clara, Calif.). Samples were injected into a Zorbax C18 column with an injection volume of 80 μL. The elution was performed with a gradient mobile phase composed of acetonitrile and water with 0.1% (v/v) trifluoroacetic acid (TFA) (8-18% acetonitrile, 0-20 min) at a flow rate of 1 mL/min. Vancomycin was detected by a UV/Vis detector at 280 nm and the detector temperature was 20° C. The acquired vancomycin intensity was compared with a linear standard curve of vancomycin at different concentrations to calculate the amount of vancomycin encapsulated inside the liposomal formulations.

To measure the toxin-triggered vancomycin release from the liposomes, the sample was mixed with PEG (100 mg/mL) and incubated with a methicillin-resistant S. aureus, strain MRSA252 (1×10⁸ CFU/mL), in 5% (v/v) tryptic soy broth (TSB) at 37° C. for 0.5 h and 24 h, respectively. After incubation, free vancomycin was separated by filtration through centrifugal filter unit (100 kDa MWCO) for 20 min at 13.2×10³ rpm. The amount of vancomycin in filtrate was quantified by HPLC following the protocol described above.

Antimicrobial Assay.

Vancomycin AuChi-Liposome were mixed with PEG (100 mg/mL) and incubated with MRSA252 (1×10⁸ CFU/mL) in 5% (v/v) TSB at 37° C. for 24 h. After incubation, the absorbance of the bacteria at 600 nm was measured by a spectrophotometer to determine bacterial growth. To exclude possible interference from background, the absorbance of the corresponding samples without MRSA252 was measured and subtracted from the obtained OD600. In the study, vancomycin loaded liposome without AuChi stabilization (Vancomycin Liposome) and free vacomycin served as positive controls, while AuChi-Liposome (without vancomycin) and PBS served as negative controls. All experiments were repeated three times.

### Results and Discussion

In order to prepare AuChi-Liposome, AuChi were first synthesized by an ex situ stabilization technique following a previously described protocol. Briefly, gold hydrosol was synthesized by sodium borohydride reduction method and then was stabilized by a calculated amount of chitosan in an ambient condition. The formation of AuChi was first confirmed by the ¹H-NMR spectroscopy. As shown in FIG. 12A, the characteristic proton resonance of chitosan was significantly shifted towards upfield when chitosan was attached to gold nanoparticles. For example, in the spectrum of free chitosan, the protons at α-carbon (anomeric carbon, C-1) with a resonance peak at 4.8 ppm was completely masked by the broad D₂O resonance; the protons at β-carbon (C-2 carbon) showed a resonance peak at 2.9 ppm; and all other glycosidic protons were centered at 3.3 to 3.8 ppm. In contrast, in the ¹H-NMR spectrum of AuChi, both α and β protons were shifted from 4.8 to 4.3 ppm and 2.9 to 2.5 ppm, respectively. In addition, the broad peaks centered at 3.3 to 3.8 ppm corresponding to the glycosidic protons of chitosan were significantly shifted towards upfield and centered at 2.6 to 3.5 ppm. This significant shifting of proton towards upfield can be attributed to their close proximity to the metal center and the inhomogeneity created by metal center, which further confirms the formation of AuChi. Similar shifting of proton resonance in close proximity to the metal center has been previously observed on different amino acid capped gold nanoparticles. The formation of AuChi was further confirmed by UV-Vis spectroscopy. As shown in FIG. 12B, AuChi exhibit a strong absorbance at 512 nm, characteristic of the corresponding bare gold nanoparticles without chitosan coating. This indicates that the coating of chitosan did not alter the plasmon resonance of gold nanoparticles. The morphology of the AuChi particles was imaged by scanning transmission electron microscope (STEM). Secondary electron (SE) signal, which provides surface topology detail, showed ˜10 nm size of AuChi with nearly uniform size distribution. The direct transmitted electron (TE) signal showed ˜4 nm size of the inner gold core, which was consistent with the size of unmodified gold nanoparticles. Based on both the SE and TE images (FIG. 12B insets), we conclude that the increase in size from 4 nm to 10 nm was solely contributed by the coating of chitosan, but not the aggregation of gold particles.

As surface properties of AuChi are crucial for their interactions with liposomes, we next characterized the surface zeta potential of AuChi by measuring their electrophoretic mobility using dynamic light scattering (DLS). The zeta potential of AuChi was 43.4±1.0 mV, indicating the presence of cationic amine groups of chitosan on the particle surface. Subsequently, liposomes consisting of hydrogenated L-α-phosphatidylcholine (Egg PC) and cholesterol (50:50 weight ratio) were prepared by vesicle extrusion technique. In order to exclude the interference of ionic strength in surface zeta potential measurements, the liposomes were prepared in deionized water. The size and surface zeta potential of the formed liposomes were 110±1 nm and −14.1±0.4 mV, respectively (FIG. 12C). Then the AuChi-Liposomes were prepared by mixing the synthesized liposomes and AuChi at a molar ratio of 1:300 under gentle bath sonication for 10 min. The size and surface zeta potential of the resulting AuChi-Liposome complexes were characterized by DLS. The measured size of AuChi-Liposome was slightly larger than that of bare liposomes suggesting the adsorption of 10 nm AuChi onto the liposome surface. The surface zeta potential changed explicitly from −14.1±0.4 mV to 35.6±0.4 mV (FIG. 12C), which confirms the binding of positively charged AuChi to the negatively charged liposomes through electrostatic attraction.

The stability of AuChi-Liposome was evaluated by a fluorescence assay consisting of 8-aminonaphthalene-1,3,6-trisulfonic acid disodium salt (ANTS) and p-xylene-bis-pyridinium bromide (DPX). ANTS is a polyanionic fluorophore and DPX is a corresponding cationic quencher. This pair of fluorophore/quencher has been widely used to study liposomal leakage upon liposome fusion with one another or with other biological membranes and thus to evaluate the stability of liposomes. When these two dyes are co-encapsulated inside liposomes at a proper molar ratio, the fluorescence emission of ANTS can be maximally quenched by DPX through a collisional quenching effect. However, when the dye-loaded liposomes are not stable and fuse with other substances, the dyes will leach out of the liposomes and be diluted by the surrounding medium. The dilution will reduce the chance of collision between ANTS and DPX and then lead to fluorescence recovery of ANTS. Therefore, with an excitation at 360 nm, ANTS emission signal at 510 nm is typically used to test the stability of liposomes. For instance, FIG. 13A shows the fluorescence emission signal of ANTS/DPX loaded liposomes in PBS and in the presence of 1% Triton X 100 surfactant, respectively. It was clearly seen that negligible signal from ANTS was detected when the liposomes are intact in PBS buffer, but a significant signal increase occurred in the presence of a membrane pore-forming surfactant such as Triton X-100. The stability of AuChi-Liposome complex was tested at various liposome/AuChi molar ratios (e.g., 1:0, 1:150, and 1:300). The AuChi-Liposome were pre-loaded with ANTS and DPX and then each sample was incubated with bare liposomes at the molar ratio of 1:4 for 1 h. The bare liposomes were neither stabilized with AuChi nor loaded with the dye pair. If fusion between AuChi-Liposome and bare liposomes occurs, it is expected that some of the dyes will transfer from AuChi-Liposome to bare liposomes. To amplify the signal of the transferred dyes, the sample was centrifuged through a filter membrane at 13.2×10³ rpm for 20 min, at which condition both bare liposomes and unstable AuChi-Liposome were ruptured and completely released the dyes, while stable AuChi-Liposome remain intact. Therefore, the fluorescence intensity of ANTS detected in the filtrate was the accumulative signal from unstable AuChi-Liposomes that have fused with either bare liposomes or filter membrane. As shown in FIG. 13B, high level of ANTS signal was detected when the liposomes were not protected by any AuChi. In contrast, when the liposome/AuChi molar ratio was 1:150 and 1:300, the detected ANTS signal was only 30% and 20%, respectively, of the bare liposomes. The obtained ANTS signal at low liposome/AuChi molar ratios (e.g., 1:150 and 1:300) may be attributed to incomplete quenching of ANTS by DPX. The collisional quenching mechanism of this pair of dyes determines that the fluorescence quenching is neither permanent nor complete. These results demonstrate that the absorption of AuChi on liposome surface can effectively prevent them from fusion with one another or filter membranes under rigorous centrifugation and thus significantly improve the stability of the liposomes. These results are also consistent with previous stability study using negatively charged carboxyl-modified gold nanoparticle to stabilize cationic liposomes (Pornpattananangkul, D et al. ACS Nano 2010, 4, 1935-1942). As liposome/AuChi molar ratio of 1:300 gave the most stable formulation, this formulation was selected for the subsequent toxin-triggered drug release studies.

With the liposome:AuChi molar ratio fixed, the liposome formulation was further optimized to obtain the highest pore forming property by bacterial toxin, α-toxin in particular. Alpha-toxin is one of the pore-forming toxins secreted by S. aureus bacterium and also the most commonly reported toxin to form pores in artificial or biological membranes. To find an optimal liposome formulation that is the most sensitive to α-toxin, two parameters were investigated; the content of cholesterol in liposome membranes and the addition of polyethylene glycols (PEG) to the liposome solutions. Both parameters have been previously reported to affect the pore-forming activity of toxins in artificial membranes. In this Example, ANTS/DPX dyes containing liposomes with different cholesterol levels (e.g., 0 wt %, 10 wt %, 25 wt %, and 50 wt %) were prepared and then incubated with α-toxin (20 μg/mL) for 1 h prior to measuring the fluorescence emission of ANTS. Maximal dye leakage was obtained by lysing all liposomes with 1% (v/v) Triton X-100, while fluorescence emission of the dyes from corresponding liposomes in PBS served as background signal. Percentage of pore forming by α-toxin was calculated using the formula: Percentage of pore forming (%)=(I_{α-toxin}−I_{PBS})/(I_{TX-100}−I_{PBS})×100, in which I_{α-toxin}, I_{PBS}, and I_{TX-100} represent fluorescence emission intensity at 510 nm of the liposome formulations incubated with α-toxin, PBS, and Triton-X-100, respectively. As shown in FIG. 14A, increase in pore forming was observed when cholesterol content increased, suggesting that cholesterol augments the pore forming efficiency of α-toxin. It was found that 50 wt % of cholesterol in the liposome membrane allowed maximal pore forming activity of α-toxin. It has been hypothesized that cholesterol can promote the interaction between α-toxin and phosphatidylcholine headgroup or interact with α-toxin itself. Next we fixed the cholesterol concentration at 50 wt % in the liposome formulation and investigated the effects of PEG on the pore forming activity of α-toxin. ANTS/DPX containing liposomes were first mixed with PEG at different PEG concentrations ranging from 0 to 150 mg/mL and then incubated with α-toxin for 1 h, followed by quantifying the percentage of pore forming. As shown in FIG. 14B, when PEG concentration in the solution increased from 0 to 100 mg/mL, pore forming increased and then reached the maximum at 100 mg/mL. However, the pore forming dropped when the PEG concentration was higher than 100 mg/mL. The role of PEG is to dehydrate liposome surfaces because of its strong hydrogen bonding with water, and thus to facilitate the membrane insertion process of toxins. These results suggest that the most sensitive liposome formulation to α-toxin contains 50% cholesterol in the liposome membrane and 100 mg/mL PEG in the solution.

Once the toxins insert into the membrane, the assembled protein oligomers are stable over a wide range of pH and temperature and the formed transmembrane pores stay open at normal conditions. Through these pores, drug payloads can be released from the liposomes. In order to verify that using toxins to form pores and trigger the release of drugs from AuChi-Liposome, we chose MRSA as a bacterium model that secretes toxins and vancomycin as an antibiotic model that has strong inhibitory effects against MRSA bacteria. In this Example, optimal formulation of AuChi-Liposome determined from the above studies were loaded with 10 mg/mL of vancomycin and incubated with MRSA252 bacteria (1×10⁸ CFU/mL) in 5% tryptic soy broth (TSB) at 37° C. At predetermined time points, released vancomycin was collected from the mixture solution using a centrifugal filter unit with a molecular weight cut-off of 100 KDa. The concentration of vancomycin was determined by reversed phase HPLC. In the experiment, the final vancomycin concentration was about 62 μg/mL. As the minimal inhibitory concentration (MIC) of vancomycin against MRSA bacteria is about 2 μg/mL, it is provided that the amount of vancomycin absorbed by cell membranes will not significantly affect the measurement of vancomycin release kinetics. In this example, the UV absorbance intensity at 280 nm was measured for a series of vancomycin samples ranging from 0-100 μg/mL to generate a standard curve (FIG. 15, inset). Then the concentration of the released vancomycin was quantified by comparing the measured absorbance intensity with the standard curve. As shown in FIGS. 15, at 0.5 h and 24 h post incubation of vancomycin-loaded AuChi-Liposome with MRSA bacteria, 29.5 μg/mL and 62.0 μg/mL of vancomycin were detected in the release medium, which translate to accumulative drug release of 48% and 100% of the total encapsulated vancomycin, respectively. In contrast, no free vancomycin was detected at either time point when the vancomycin-loaded AuChi-Liposomes were incubated in the absence of MRSA bacteria. This further confirms that AuChi-Liposome remained stable during the centrifugation process and thus the vancomycin detected in the presence of MRSA was solely contributed by the bacterial toxins through forming pores on liposome membranes. Since 24 h are a standard incubation time to study antimicrobial activity of antibiotics, complete drug release from vancomycin-loaded AuChi-Liposome obtained at this time point implies the potential application of this system to efficiently suppress bacterial growth.

After having demonstrated the drug release from AuChi-Liposome in the presence of toxins secreted by MRSA bacteria, the ability of vancomycin-loaded AuChi-Liposome to inhibit the growth of MRSA252 in vitro was examined. Vancomycin-loaded AuChi-Liposomes were incubated with MRSA252 (1×10⁸ CFU/mL) in 5% TSB for 24 h, followed by OD₆₀₀ measurement to determine the bacterial growth. Vancomycin-loaded liposomes without AuChi stabilization and free vancomycin were used as positive controls; blank AuChi-Liposome (without vancomycin) and PBS served as negative controls. As shown in FIG. 16, vancomycin AuChi-Liposomes were able to inhibit the growth of MRSA252 to the same extent as vancomycin liposomes and free vancomycin. The student t-test showed that the difference between the OD₆₀₀ value of vancomycin AuChi-liposome and that of vancomycin was insignificant with a p-value of 0.18 (p>0.1). The obtained OD₆₀₀ signal of vancomycin AuChi-liposome has subtracted that of AuChi-liposome (without vancomycin) to exclude any possible interference signal from the bare liposomal drug carriers. The observed non-negligible inhibitory effects of AuChi-liposome in FIG. 16 might be due to some intrinsic properties of lipids and/or the interactions between unbound AuChi nanoparticles and the bacteria. Although both vancomycin AuChi-Liposome and vancomycin liposome inhibited the growth of MRSA252 bacteria, their working mechanisms were different. Vancomycin AuChi-Liposome were stabilized against fusion and did not release drugs in the absence of bacterial toxins. Thus, their observed inhibitory effect was merely due to the released vancomycin through the pores formed by bacterial toxins. In contrast, vancomycin liposome was not protected by AuChi and could readily fuse with each other and bacterial membranes resulting in vacomycin release, which answered for the observed inhibitory effect. Comparing to bare vancomycin liposome, the vancomycin AuChi-Liposome system exhibits several distinct advantages. First, it improves the shelf-time of the liposome formulation that minimal amount of drugs will be released prior to administration. Secondly, it enables bacteria-targeted antibiotic delivery. As this formulation doesn't fuse with biological membranes, the drugs will only be released at the infectious sites where the bacteria secrete toxins. Lastly, the dosage of the antibiotics is self-regulated by the severeness of the infections. More bacteria will secrete more toxins and thus trigger more drug release. Note that the minimum inhibitory concentration (MIC) of vancomycin against MRSA is about 2 μg/mL. The released vancomycin from vancomycin AuChi-Liposome had a concentration up to 62 μg/mL, which should be sufficient to inhibit the growth of the bacteria.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention. Specifically intended to be within the scope of the present invention, and incorporated herein by reference in its entirety, is the following publications:
Dissaya Pornpattananangkul, Li Zhang, Sage Olson, Santosh Aryal, Marygorret Obonyo, Kenneth Vecchio, Chun-Ming Huang, and Liangfang Zhang, Bacterial Toxin-Triggered Drug Release from Gold Nanoparticle-Stabilized Liposomes for the Treatment of Bacterial Infection, J. Am. Chem. Soc. Articles ASAP (Feb. 23, 2011) and Pornpattananangkul D, Olson S, Aryal S, Sartor M, Huang C M, Vecchio K, Zhang L., Stimuli-responsive liposome fusion mediated by gold nanoparticles, ACS Nano. 2010 Apr. 27; 4(4):1935-42.
   Other publications incorporated herein by reference in their entirety include:
1. Aryal, S.; B, K. C. R.; Dharmaraj, N.; Bhattarai, N.; Kim, C. H.; Kim, H. Y., Spectroscopic Identification of S—Au Interaction in Cysteine Capped Gold Nanoparticles. Spectrochim. Acta A 2006, 63, 160-163.
2. Barenholz, Y., Liposome Application: Problems and Prospects. Curr. Opin. Coll. Inter. Sci. 2001, 6, 66-77.
3. Bashford, C. L.; Alder, G. M.; Fulford, L. G.; Korchev, Y. E.; Kovacs, E.; MacKinnon, A.; Pederzolli, C.; Pasternak, C. A., J. Membr. Biol. 1996, 150, 37-45.
4. Bhakdi, S.; Tranum-Jensen, J., Microbiol. Rev. 1991, 55, 733-751.
5. Blackwood, R. A.; Smolen, J. E.; Hessler, R. J.; Harsh, D. M.; Transue, A., Biochem. J. 1996, 314 (Pt 2), 469-475.
6. Boisselier, E.; Astruc, D., Gold Nanoparticles in Nanomedicine: Preparations, Imaging, Diagnostics, Therapies and Toxicity. Chem. Soc. Rev. 2009, 38, 1759-1782.
7. Castro, G. A.; Ferreira, L. A., Novel Vesicular and Particulate Drug Delivery Systems for Topical Treatment of Acne. Expert Opin. Drug Deliv. 2008, 5, 665-679.
8. Corma, A.; Diaz, U.; Arrica, M.; Fernandez, E.; Ortega, Í., Organic-Inorganic Nanospheres with Responsive Molecular Gates for Drug Storage and Release. Angew. Chem. Int. Ed. 2009, 48, 6247-6250.
9. Davis, M. E.; Chen, Z. G.; Shin, D. M., Nanoparticle Therapeutics: an Emerging Treatment Modality for Cancer. Nat. Rev. Drug Discov. 2008, 7, 771-782.
10. Farokhzad, O. C.; Cheng, J.; Teply, B. A.; Sherifi, I.; Jon, S.; Kantoff, P. W.; Richie, J. P.; Langer, R., Proc. Natl. Acad. Sci. USA 2006, 103, 6315-6320.
11. Ford, S.; Menestrina, G., Eur. J. Biochem. 1989, 181, 767-773.
12. Greenman, J., Follicular pH and the Development of Acne. Int. J. Dermatol. 1981, 20, 656-658.
13. Ha, T., Single-Molecule Fluorescence Resonance Energy Transfer. Methods 2001, 25, 78-86.
14. Haluska, C. K.; Riske, K. A.; Marchi-Artzner, V.; Lehn, J.-M.; Lipowsky, R.; Dimova, R., Time Scales of Membrane Fusion Revealed by Direct Imaging of Vesicle Fusion with High Temporal Resolution. Proc. Natl. Acad. Sci. USA 2006, 103, 15841-15846.
15. Holland, D. B.; Cunliffe, W. J., Skin Surface and Open Comedone pH in Acne Patients. Acta Derm. Venereol. 1983, 63, 155-158.
16. Hu, C. M.; Kaushal, S.; Tran Cao, H. S.; Aryal, S.; Sartor, M.; Esener, S.; Bouvet, M.; Zhang, L., Mol. Pharm. 2010, 7, 914-920.
17. Kim, H. J.; Jones, M. N., J. Liposome Res. 2004, 14, 123-139.
18. Kostarelos, K.; Tadros, T. F.; Luckham, P. F., Physical Conjugation of (Ttri-) Block Copolymers to Liposomes Toward the Construction of Sterically Stabilized Vesicle Systems. Langmuir 1999, 15, 369-376.
19. Langer, R., Nature 1998, 392 (SUPP), 5-10.
20. Lei, G.; MacDonald, R. C., Lipid Bilayer Vesicle Fusion: Intermediates Captured by High-Speed Microfluorescence Spectroscopy. Biophys. J. 2003, 85, 1585-1599.
21. Lian, T.; Ho, R. J. Y., Trends and Developments in Liposome Drug Delivery Systems. J. Pharma. Sci. 2001, 90, 667-680.
22. Liptak, M. D.; Shields, G. C., Accurate pK(a) Calculations for Carboxylic Acids Using Complete Basis Set and Gaussian Models Combined with CPCM Continuum Solvation Methods. J. Am. Chem. Soc. 2001, 123, 7314-7319.
23. Liu, R.; Kay, B. K.; Jiang, S.; Chen, S., MRS Bulletin 2009, 34, 432-440.

24. Marrink, S.; Mark, A. E., The Mechanism of Vesicle Fusion as Revealed by Molecular Dynamics Simulations. J. Am. Chem. Soc. 2003, 125, 11144-11145.
25. Mayer, L. D.; Hope, M. J.; Cullis, P. R., Vesicles of Variable Sizes Produced by a Rapid Extrusion Procedure. Biochim. Biophys. Acta 1986, 858, 161-168.
26. Meesters, C.; Brack, A.; Hellmann, N.; Decker, H., Proteins 2009, 75, 118-126.
27. Moghimi, S. M.; Szebeni, J., Stealth Liposomes and Long Circulating Nanoparticles: Critical Issues in Pharmacokinetics, Opsonization and Protein-Binding Properties. Prog. Lipid Res. 2003, 42, 463-478.
28. Okumus, B.; Arslan, S.; Fengler, S. M.; Myong, S.; Ha, T., J. Am. Chem. Soc. 2009, 131, 14844-14849.
29. Patil, V.; Malvankar, R. B.; Sastry, M., Role of Particle Size in Individual and Competitive Diffusion of Carboxylic Acid Derivatized Colloidal Gold Particles in Thermally Evaporated Fatty Amine Films. Langmuir 1999, 15, 8197-8206.
30. Pornpattananangkul, D.; Olson, S.; Aryal, S.; Sartor, M.; Huang, C. M.; Vecchio, K.; Zhang, L., ACS Nano 2010, 4, 1935-1942.
31. Prausnitz, M. R.; Langer, R., Transdermal Drug Delivery. Nat. Biotech. 2008, 26, 1261-1268.
32. Ringsdorf, H.; Schlarb, B.; Venzmer, J., Molecular Architecture and Function of Polymeric Oriented Systems—Models for the Study of Organization, Surface Recognition, and Dynamics of Biomembranes. Angew. Chem. Int. Ed. 1988, 27, 113-158.
33. Sakoulas, G.; Moise-Broder, P. A.; Schentag, J.; Forrest, A.; Moellering, R. C., Jr.; Eliopoulos, G. M., J. Clin. Microbiol. 2004, 42, 2398-2402.
34. Schafer-Korting, M.; Mehnert, W.; Korting, H. C., Lipid Nanoparticles for Improved Topical Application of Drugs for Skin Diseases. Adv. Drug Deliv. Rev. 2007, 59, 427-443.
35. Schmid-Wendtner, M. H.; Korting, H. C., The pH of the Skin Surface and Its Impact on the Barrier Function. Skin Pharmacol. Physiol. 2006, 19, 296-302.
36. Semple, S. C.; Chonn, A.; Cullis, P. R., Influence of Cholesterol on the Association of Plasma Proteins with Liposomes. Biochemistry 1996, 35, 2521-2525.
37. Shen, X.; Valencia, C. A.; Szostak, J. W.; Dong, B.; Liu, R., Proc. Natl. Acad. Sci. USA 2005, 102, 5969-5974.
38. Shi, J.; Votruba, A. R.; Farokhzad, O. C.; Langer, R., Nano Lett. 2010, 10, 3223-3230.

39. Shukla, R.; Bansal, V.; Chaudhary, M.; Basu, A.; Bhonde, R. R.; Sastry, M., Biocompatibility of Gold Nanoparticles and Their Endocytotic Fate Inside the Cellular Compartment: A Microscopic Overview. Langmuir 2005, 21, 10644-10654.
40. Sinico, C.; Fadda, A. M., Vesicular Carriers for Dermal Drug Delivery. Expert Opin. Drug Deliv. 2009, 6, 813-825.
41. Song, L.; Hobaugh, M. R.; Shustak, C.; Cheley, S.; Bayley, H.; Gouaux, J. E., Science 1996, 274, 1859-1866.
42. Stockler, S.; Lackner, H.; Ginter, G.; Schwinger, W.; Plecko, B.; Muller, W., Eur. J. Pediatr. 1993, 152, 981-983.
43. Torchilin, V. P., Recent Advances with Liposomes as Pharmaceutical Carriers. Nat. Rev. Drug Discov. 2005, 4, 145-160.
44. Vecsey-Semjen, B.; Kwak, Y. K.; Hogbom, M.; Mollby, R., J. Membr. Biol. 2010, 234, 171-181.
45. Walsh, T. J.; Goodman, J. L.; Pappas, P.; Bekersky, I.; Buell, D. N.; Roden, M.; Barrett, J.; Anaissie, E. J., Antimicrob. Agents Chemother. 2001, 45, 3487-3496.
46. Wang, A. Z.; Gu, F.; Zhang, L.; Chan, J. M.; Radovic-Moreno, A.; Shaikh, M. R.; Farokhzad, O. C., Expert Opin. Biol. Ther. 2008, 8, 1063-1070.
47. Wang, B.; Zhang, L.; Bae, S. C.; Granick, S., Nanoparticle-Induced Surface Reconstruction of Phospholipid Membranes. Proc. Natl. Acad. Sci. USA 2008, 105, 18171-18175.
48. Watanabe, M.; Tomita, T.; Yasuda, T., Biochim. Biophys. Acta 1987, 898, 257-265.
49. Woodle, M. C., Adv. Drug Deliv. Rev. 1998, 32, 139-152.
50. Woodle, M. C., Controlling Liposome Blood Clearance by Surface-Grafted Polymers. Adv. Drug Deliv. Rev. 1998, 32, 139-152.
51. Wyman, T. B.; Nicol, F.; Zelphati, 0.; Scaria, P. V.; Plank, C.; Szoka, F. C., Biochemistry 1997, 36, 3008-3017.
52. Xu, D.; Cheng, Q., J. Am. Chem. Soc. 2002, 124, 14314-14315.
53. Yang, D.; Pornpattananangkul, D.; Nakatsuji, T.; Chan, M.; Carson, D.; Huang, C. M.; Zhang, L., The Antimicrobial Activity of Liposomal Lauric Acids against Propionibacterium acnes. Biomaterials 2009, 30, 6035-6040.
54. Yu, Y.; Anthony, S. M.; Zhang, L.; Bae, S. C.; Granick, S., J. Phys. Chem. C 2007, 111, 8233-8236.
55. Yu, Y.; Vroman, J. A.; Bae, S. C.; Granick, S., J. Am. Chem. Soc. 2010, 132, 195-201.
56. Zhang, L.; Granick, S., How to Stabilize Phospholipid Liposome (Using Nanoparticles). Nano Lett. 2006, 6, 694-698.
57. Zhang, L.; Gu, F. X.; Chan, J. M.; Wang, A. Z.; Langer, R. S.; Farokhzad, O. C., Nanoparticles in Medicine: Therapeutic Applications and Developments. Clin. Pharmacol. Ther. 2008, 83, 761-769.
58. Zhang, L.; Hong, L.; Yu, Y.; Bae, S. C.; Granick, S., Nanoparticle-Assisted Surface Immobilization of Phospholipid Liposomes. J. Am. Chem. Soc. 2006, 128, 9026-9027.
59. Zhang, L.; Pornpattananangkul, D.; Hu, C.-M. J.; Huang, C.-M., Curr. Med. Chem. 2010, 17, 585-594.

What is claimed is:

1. A liposome comprising an inner sphere and an outer surface of the liposome, a plurality of biocompatible nanoparticles, said biocompatible nanoparticles connected to the lipid molecules with a stimuli-sensitive bond, and further comprising a cargo within the inner sphere, wherein said cargo is released upon triggering the stimuli-sensitive bond.

2. The liposome according to claim 1, wherein the biocompatible nanoparticles are selected from the group consisting of gold nanoparticles, silver nanoparticles, and synthetic nanoparticles.

3. The liposome according to claim 1, wherein the surface of the biocompatible nanoparticles comprises anionic functional groups.

4. The liposome according to claim 1, wherein the surface of the biocompatible nanoparticles comprises cationic functional groups.

5. The liposome according to claim 1, wherein the surface of the biocompatible nanoparticle comprises carboxylates.

6. The liposome according to claim 1, wherein the biocompatible nanoparticle is about 1 to about 20 nm in diameter.

7. The liposome according to claim 1, wherein the liposome comprises hydrogenated L-α-phosphatidylcholine and 1,2-di-(9Z-octadecenoyl)-3-trimethylammoniumpropane.

8. The liposome according to claim 1, wherein the cargo is selected from the group consisting of antibiotics, antimicrobials, growth factors, chemotherapeutic agents, and combinations thereof.

9. The liposome according to claim 1, wherein the cargo is selected from the group consisting of lauric acid, benzoyl peroxide, vancomycin, and combinations thereof.

10. The liposome according to claim 1, wherein the liposome is about 10 to about 300 nm in diameter.

11. The liposome according to claim 1, wherein the biocompatible nanoparticles comprise about 5 to about 25% of the liposome surface.

12. The liposome according to claim 1, wherein the trigger is selected from the group consisting of dermal pH, naturally-occurring or synthetic toxin pore forming activity, and light administration.

13. The liposome according to claim 1, wherein the stimuli-sensitive bond is a pH-sensitive bond.

14. A liposome comprising an inner sphere and an outer surface of the liposome, a plurality of biocompatible nanoparticles, said biocompatible nanoparticles being in contact with the lipid molecules via electrostatic interaction, and further comprising a cargo within the inner sphere, wherein said cargo is released upon triggering liposome pore formation.

15. The liposome according to claim 14, wherein the biocompatible nanoparticles are selected from the group consisting of gold nanoparticles, silver nanoparticles, and synthetic nanoparticles.

16. The liposome according to claim 14, wherein the surface of the biocompatible nanoparticles comprises anionic functional groups.

17. The liposome according to claim 14, wherein the surface of the biocompatible nanoparticles comprises cationic functional groups.

18. The liposome according to claim 14, wherein the surface of the biocompatible nanoparticle comprises chitosan.

19. The liposome according to claim 14, wherein the biocompatible nanoparticle is about 1 to about 20 nm in diameter.

20. The liposome according to claim 14, wherein the liposome comprises hydrogenated L-α-phosphatidylcholine and 1,2-di-(9Z-octadecenoyl)-3-trimethylammoniumpropane.

21. The liposome according to claim 14, wherein the cargo is selected from the group consisting of antibiotics, antimicrobials, growth factors, chemotherapeutic agents, and combinations thereof.

22. The liposome according to claim 14, wherein the cargo is selected from the group consisting of lauric acid, benzoyl peroxide, vancomycin, and combinations thereof.

23. The liposome according to claim 14, wherein the liposome is about 10 to about 300 nm in diameter.

24. The liposome according to claim 14, wherein the bound gold nanoparticles comprise about 5 to about 25% of the liposome surface.

25. The liposome according to claim 14, wherein the trigger is selected from the group consisting of dermal pH, naturally-occurring or synthetic toxin pore forming activity, and UV light administration.

26. The liposome according to claim 14, wherein the liposome comprises 50% cholesterol in the membrane and 100 mg/mL PEG in the solution.

27. A medicament delivery system comprising a composition of claim 1.

28. The medicament delivery system of claim 27, in a pharmaceutically acceptable vehicle.

29. A method of selectively delivering cargo to target dermal sites, the method comprising administering a liposome of claim 1 to the target dermal site and triggering cargo release.

30. A method for treating a dermal disease or condition, the method comprising administering a therapeutically effective amount of a liposome of claim 1 to a target dermal site of a subject in need thereof and triggering cargo release.

31. The method of claim 30, wherein the condition is selected from the group consisting of MRSA infection, S. aureus infection, and P. acnes infection.

32. A method of stably storing medicaments prior to triggered release, the method comprising enclosing the medicaments in a liposome of claim 1.
